(19) United States
(12) Patent Application Publication
(10) Pub. No.: US 2026/0183423 A1
(43) Pub. Date: Jul. 2, 2026

(54) CATIONIC LIPID HAVING AROMATIC ESTER BONDS

(71) Applicants: NOF CORPORATION, Tokyo (JP); TOHOKU UNIVERSITY, Sendai-shi (JP)

(72) Inventors: Yuhei ONIZUKA, Kawasaki (JP); Yuki AOTA, Kawasaki (JP); Ryosuke MOTOUSU, Kawasaki (JP); Tokihiro TANAKA, Kawasaki (JP); Kasumi ITO, Kawasaki (JP); Naoya ICHIHARA, Kawasaki (JP); Yuta NAKAI, Kawasaki (JP); Kota TANGE, Kawasaki (JP); Hidetaka AKITA, Sendai (JP); Hiroki TANAKA, Sendai (JP)

(73) Assignees: NOF CORPORATION, Tokyo (JP); TOHOKU UNIVERSITY, Sendai-shi (JP)

(21) Appl. No.: 19/432,289

(22) Filed: Dec. 24, 2025

(30) Foreign Application Priority Data
Dec. 27, 2024 (JP) 2024-231975

(51) Int. Cl. A61K 48/00; C07C 323/12; C07D 207/09; C07D 207/325; C07D 209/14; C07D 213/36; C07D 233/64; C07D 295/13

(52) U.S. Cl. CPC A61K 48/0033; C07C 323/12; C07D 207/09; C07D 207/325; C07D 209/14; C07D 213/36; C07D 233/64; C07D 295/13

(57) ABSTRACT

A cationic lipid represented by the following formula (1) (wherein symbols are as described in the SPECIFICATION) that may be used as a nucleic acid delivery carrier with good nucleic acid delivery efficiency is provided.

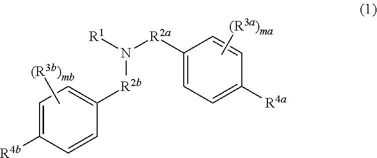

### CROSS REFERENCE TO RELATED APPLICATION

This application claims priority to Japanese Patent Application No. patent application No. 2024-231975, filed on Dec. 27, 2024, which is incorporated herein by reference in its entirety.

### BACKGROUND OF THE INVENTION

### Field of the Invention

The present invention relates to a cationic lipid having aromatic ester bonds, a lipid membrane structure containing same, a nucleic acid-introducing agent and a pharmaceutical composition containing any of these, a method for introducing a nucleic acid into a cell or a target cell, and a production method of cellular medicines. In the present specification, the “aromatic ester bond” means an ester bond bonded to an aromatic ring.

### Discussion of the Background

For practicalization of nucleic acid therapy, an effective and safe nucleic acid delivery carrier is demanded. While virus vectors are nucleic acid delivery carriers with good nucleic acid delivery efficiency, the development of non-viral nucleic acid delivery carriers that can be used more safely is ongoing. Among them, carriers using a cationic lipid are non-viral nucleic acid delivery carriers most generally used at present.

Cationic lipids are largely composed of an amine moiety and a lipid moiety. In cationic lipids, the amine moiety showing cationicity and a polyanion nucleic acid electrostatically interact to form a liposome or lipid membrane structure, which promotes uptake into cells and delivers the nucleic acid into cells.

As known cationic lipids generally and widely used, 1,2-dioleoyloxy-3-trimethylammonium-propane (DOTAP) and 1,2-dioleoyloxy-3-dimethylaminopropane (DODAP) can be mentioned. These known cationic lipids form a positively-charged liposome or lipid membrane structure when combined with a phospholipid, which electrostatically interacts with a nucleic acid to be able to deliver the nucleic acid to the target cells (Biomaterials 29 (24-25): 3477-3496, 2008).

On the other hand, for a lipid membrane structure using a cationic lipid to exhibit a practical effect in vivo as a nucleic acid delivery carrier, it needs to show specific pharmacokinetics. To be precise, requirements such as high stability in blood and the like need to be fulfilled. To address this problem, it is known that a lipid membrane structure in which pKa of the surface of the lipid membrane structure is adjusted to near neutral and PEG-lipid is introduced exhibits a long life in blood after intravenous injection and accumulates at tumor sites. Furthermore, there is an example of improving pharmacokinetics by adjusting surface pKa of lipid membrane structure. There is also an example of improving pharmacokinetics by controlling the particle size of lipid membrane structure.

For example, Molecular Therapy 24(4): 788-795, 2016 and Angewante Chemie International Edition 51: 8529-8533, 2012 show that pharmacokinetics and distribution in each cell in the liver can be controlled by adjusting the surface pKa of lipid membrane structures. These literatures show that escape of lipid membrane structures from endosomes is promoted and nucleic acids can be efficiently delivered into the cytoplasm by adjusting the surface pKa of lipid membrane structures for endosome escape.

Pharmaceutics 2024, 16(12), 1521 shows that the particle size of lipid membrane structures plays an important role in various biological phenomena, such as circulation half-life, extravasation, macrophage uptake, biodistribution, and the like. In particular, Pharmaceutics 2024, 16(12), 1521 shows that a high transfection effect can be achieved in in vitro tests by controlling the particle size of lipid membrane structures to 20 to 200 nm.

### SUMMARY OF THE INVENTION

While cationic lipids having improved pharmacokinetics have been developed as mentioned above, in view of the property of the nucleic acid delivery carriers that they generally introduce exogenous substances into cells, a large effect output from a small uptake amount is desired. That is, when a lipid membrane structure is used as a delivery carrier of a nucleic acid (expression vector) into cells, it is desired to increase the nucleic acid delivery efficiency, thereby enhancing intracellular gene expression efficiency.

The present invention has been made in view of the above-mentioned problems, and aims to provide a cationic lipid that can produce a lipid membrane structure with good nucleic acid delivery efficiency.

The present inventors have conducted intensive studies and found that a lipid membrane structure with good nucleic acid delivery efficiency may be produced by using the below-mentioned cationic lipid of the present invention.

The present invention capable of achieving the above-mentioned object is as follows.

[1] A cationic lipid represented by the formula (1):

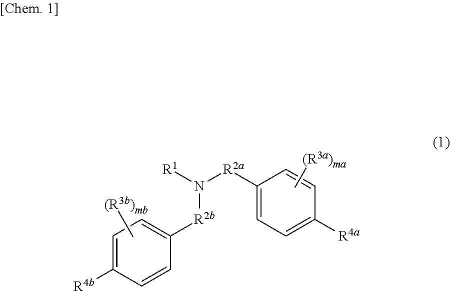

wherein
R¹ is an alkyl group having 1 to 4 carbon atoms and a nitrogen-containing heterocyclic group, a tertiary amino group, or a hydroxy group as a substituent,
R^{2a} and R^{2b} are each independently an alkylene group having 1 to 16 carbon atoms and containing at least one degradable bond,
R^{3a} and R^{3b} are each independently an alkoxy group having 1 to 4 carbon atoms or a halogen atom,
ma and mb are each independently an integer of 0 to 4,
R^{4a} and R^{4b} are each independently R⁵—CO—O—* (wherein * is a bonding position), and
R⁵ is a monovalent aliphatic hydrocarbon group having 3 to carbon atoms and optionally containing at least one degradable bond, or R⁶—CO—(CH₂)ₚ—* (wherein * is a bonding position, R⁶ is a residue of a liposoluble vitamin having a hydroxyl group or a residue of a sterol derivative having a hydroxyl group, and p is an integer of 1 to 8).
   [2] The cationic lipid of the aforementioned [1], wherein the nitrogen-containing heterocyclic group is a pyrrolidinyl group, a morpholinyl group, a piperazinyl group, a piperidyl group, an azepanyl group, a pyrrolyl group, a pyrazolyl group, a pyridyl group, or an indolyl group,
the tertiary amino group is a di(alkyl)amino group, and
the carbon numbers of the two alkyl groups in the di(alkyl)amino group are each independently 1 to 4.
   [3] The cationic lipid of the aforementioned [1] or [2], wherein the R^{2a} and R^{2b} are each independently a divalent group represented by the formula (2):

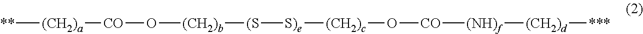

wherein
** is a bonding position to the nitrogen atom in the formula (1),
*** is a bonding position to the carbon atom of the benzene ring in the formula (1),
a to d are each independently an integer of 1 to 4, and
e and f are each independently 0 or 1.
   [4] The cationic lipid of any one of the aforementioned [1] to [3], wherein ma and mb are each 0.
   [5] The cationic lipid of any one of the aforementioned [1] to [4], wherein R⁵ is a monovalent aliphatic hydrocarbon group having 3 to 30 carbon atoms and optionally containing at least one degradable bond.
   [6] A lipid membrane structure comprising the cationic lipid of any one of the aforementioned [1] to [5] as a constituent lipid of the membrane.
   [7] The lipid membrane structure of the aforementioned [6], further comprising a nucleic acid.
   [8] A nucleic acid-introducing agent comprising the cationic lipid of any one of the aforementioned [1] to [5].
   [9] The nucleic acid-introducing agent of the aforementioned [8], further comprising a nucleic acid.
   [10] A pharmaceutical composition comprising the cationic lipid of any one of the aforementioned [1] to [5].
   [11] The pharmaceutical composition of the aforementioned [10], further comprising a nucleic acid.
   [12] A method for introducing a nucleic acid in a nucleic acid-introducing agent into a cell in vitro, comprising bringing the nucleic acid-introducing agent of the aforementioned [9] into contact with the cell.
   [13] A method for introducing a nucleic acid in a nucleic acid-introducing agent into a target cell in a living organism, comprising administering the nucleic acid-introducing agent of the aforementioned [9] to the living organism.
   [14] A method for producing a cellular medicine comprising a cell expressing a gene in a nucleic acid, comprising introducing the nucleic acid in a nucleic acid-introducing agent into a cell by bringing the nucleic acid-introducing agent of the aforementioned [9] into contact with the cell.

### Effect of the Invention

Using the cationic lipid of the present invention, a lipid membrane structure with good nucleic acid delivery efficiency may be produced.

### DETAILED DESCRIPTION OF THE PREFERRED EMBODIMENTS

The cationic lipid of the present invention is a cationic lipid represented by the following formula (1):

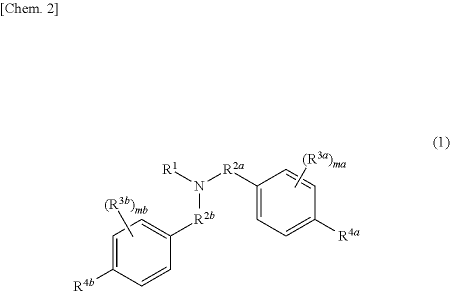

In the present specification and claims (hereinafter abbreviated as “the present specification”), the “cationic lipid represented by the formula (1)” is sometimes to be abbreviated as “cationic lipid (1)”. Groups and the like represented by other formulas are also sometimes to be abbreviated in the same manner as the “cationic lipids represented by the formula (1)”.

First, groups and the like included in the formula (1) are explained.

In the formula (1), R¹ is an alkyl group having 1 to 4 carbon atoms and having a nitrogen-containing heterocyclic group, a tertiary amino group, or a hydroxy group as a substituent. R¹ is preferably an alkyl group having 1 to 4 carbon atoms and having a nitrogen-containing heterocyclic group or a tertiary amino group as a substituent. The alkyl group having 1 to 4 carbon atoms may be linear or a branched chain. The number of carbon atoms in the aforementioned alkyl group does not include the number of carbon atoms in the nitrogen-containing heterocyclic group or tertiary amino group as a substituent.

In the present specification, the nitrogen-containing heterocyclic group may be non-aromatic or aromatic. In addition, the nitrogen-containing heterocyclic group optionally has a substituent such as an alkyl group having 1 to 4 carbon atoms and the like.

Examples of the non-aromatic nitrogen-containing heterocyclic group include pyrrolidinyl groups (e.g., a group represented by the following formula (N1) or the following formula (N2)), morpholinyl groups (e.g., a group represented by the following formula (N3)), piperazinyl groups (e.g., a group represented by the following formula (N4)), piperidyl groups (e.g., a group represented by the following formula (N5)), and azepanyl groups (e.g., a group represented by the following formula (N6)) (in the following formulas, * is a bonding position). In the present specification, “*” and the like indicate a bonding position, not a carbon atom, as described above. Therefore, for example, “—*” indicates a single bond.

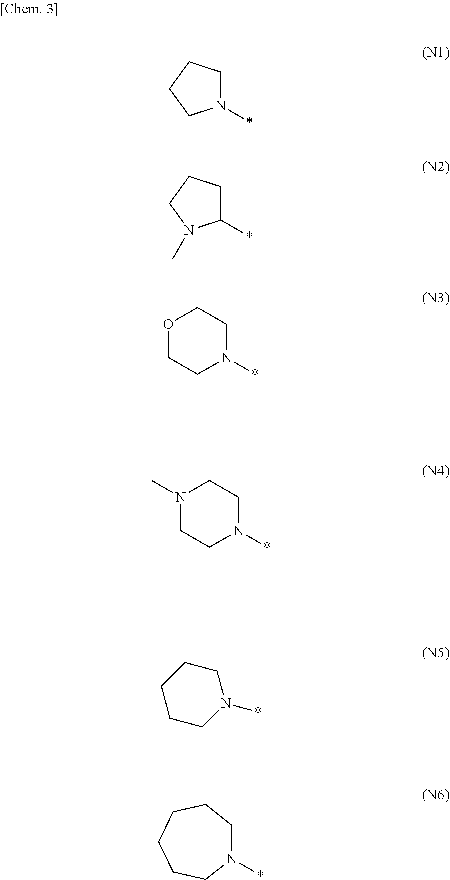

Examples of the aromatic nitrogen-containing heterocyclic group include pyrrolyl groups (e.g., a group represented by the following formula (N7)), pyrazolyl groups (e.g., a group represented by the following formula (N8)), pyridyl groups (e.g., a group represented by the following formula (N9)), and indolyl groups (e.g., a group represented by the following formula (N10)) (in the following formulas, * is a bonding position).

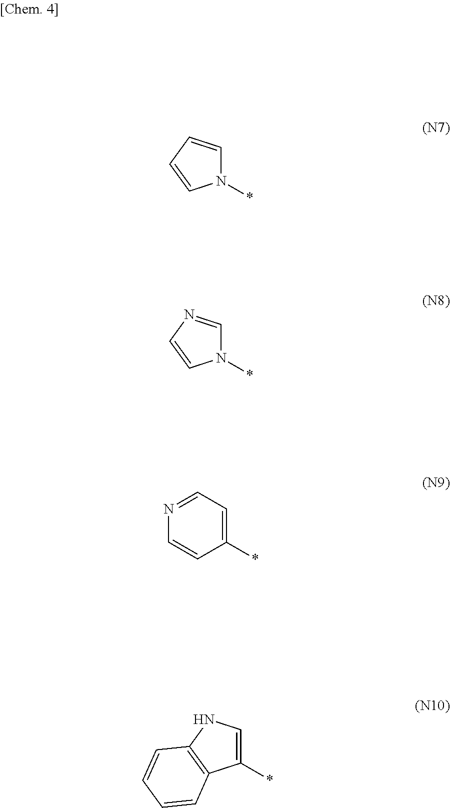 [SMILES: *n1cccc1 ; *n1ccnc1 ; *c1ccncc1 ; *c1c[nH]c2ccccc12]

In one embodiment of the present invention, the nitrogen-containing heterocyclic group is preferably a pyrrolidinyl group, a morpholinyl group, a piperazinyl group, a piperidyl group, an azepanyl group, a pyrrolyl group, a pyrazolyl group, a pyridyl group, or an indolyl group, more preferably a pyrrolidinyl group, a morpholinyl group, or an indolyl group, further preferably a pyrrolidinyl group or a morpholinyl group.

In another embodiment of the present invention, the nitrogen-containing heterocyclic group is preferably any of group (N1) to group (N10), more preferably group (N1), group (N3), or group (N10), further preferably group (N1) or group (N3).

In the present specification, examples of the tertiary amino group include di(alkyl)amino group. The carbon numbers of the two alkyl groups in the di(alkyl)amino group are each independently preferably 1 to 4, more preferably 1 to 3. Examples of the di(alkyl)amino group include the groups represented by the following formula (N11) to the following formula (N16) (in the following formulas, * is a bonding position).

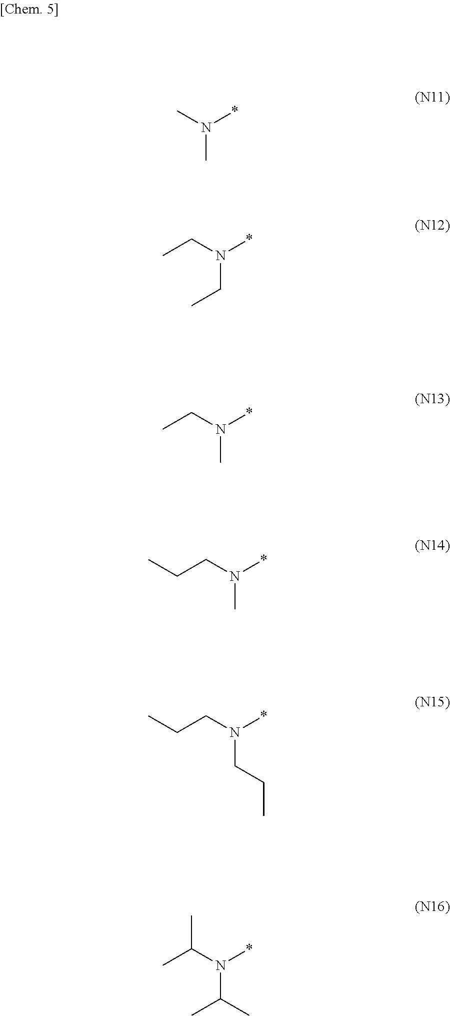

In one embodiment of the present invention, the di(alkyl)amino group is preferably any of group (N11) to group (N16), more preferably group (N12).

In one preferred embodiment of the present invention,
the nitrogen-containing heterocyclic group is a pyrrolidinyl group, a morpholinyl group, a piperazinyl group, a piperidyl group, an azepanyl group, a pyrrolyl group, a pyrazolyl group, a pyridyl group, or an indolyl group,
the tertiary amino group is a di(alkyl)amino group, and
the carbon numbers of the two alkyl groups in the di(alkyl)amino group are each independently 1 to 4.

In another preferred embodiment of the present invention,
the nitrogen-containing heterocyclic group is a pyrrolidinyl group, a morpholinyl group, or an indolyl group,
the tertiary amino group is a di(alkyl)amino group, and
the carbon numbers of the two alkyl groups in the di(alkyl)amino group are each independently 1 to 4.

In another preferred embodiment of the present invention,
R¹ is an alkyl group having 1 to 4 carbon atoms and having a nitrogen-containing heterocyclic group or a tertiary amino group as a substituent,
the nitrogen-containing heterocyclic group is a pyrrolidinyl group or a morpholinyl group,
the tertiary amino group is a di(alkyl)amino group, and
the carbon numbers of the two alkyl groups in the di(alkyl)amino group are each independently 1 to 4.

In another preferred embodiment of the present invention,
the nitrogen-containing heterocyclic group is any of group (N1) to group (N10), and
the tertiary amino group is any of group (N11) to group (N16).

In another preferred embodiment of the present invention,
the nitrogen-containing heterocyclic group is group (N1), group (N3), or group (N10), and
the tertiary amino group is group (N12).

In another preferred embodiment of the present invention,
R¹ is an alkyl group having 1 to 4 carbon atoms and having a nitrogen-containing heterocyclic group or a tertiary amino group as a substituent,
the nitrogen-containing heterocyclic group is group (N1) or group (N3), and
the tertiary amino group is group (N12).

In another preferred embodiment of the present invention, R¹ is any of a group represented by the following formula (3) to a group represented by the following formula (23) (in the following formulas, * is a bonding position).

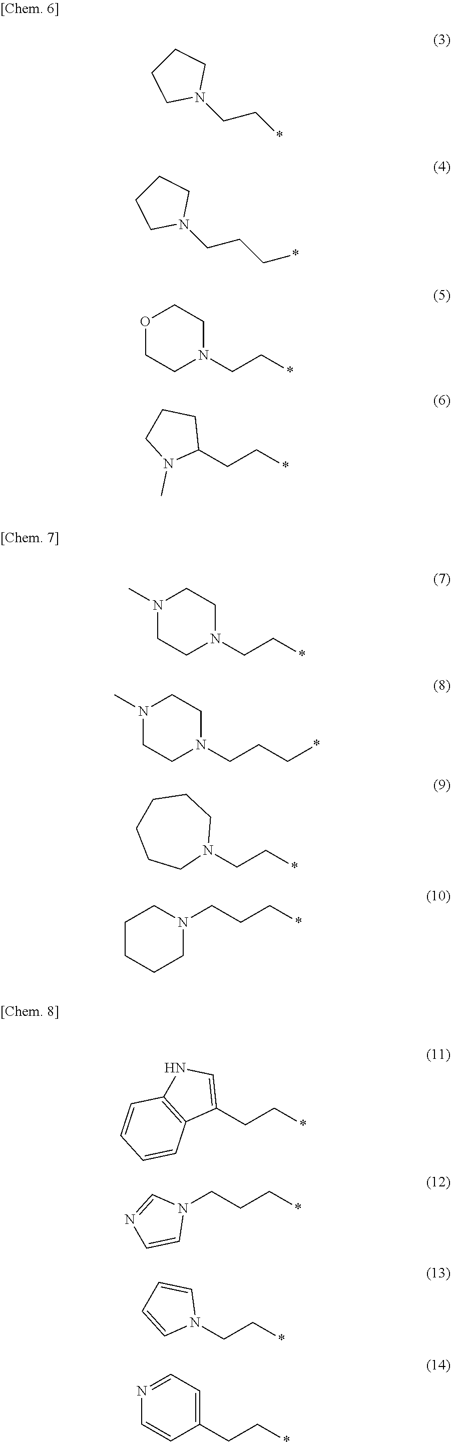

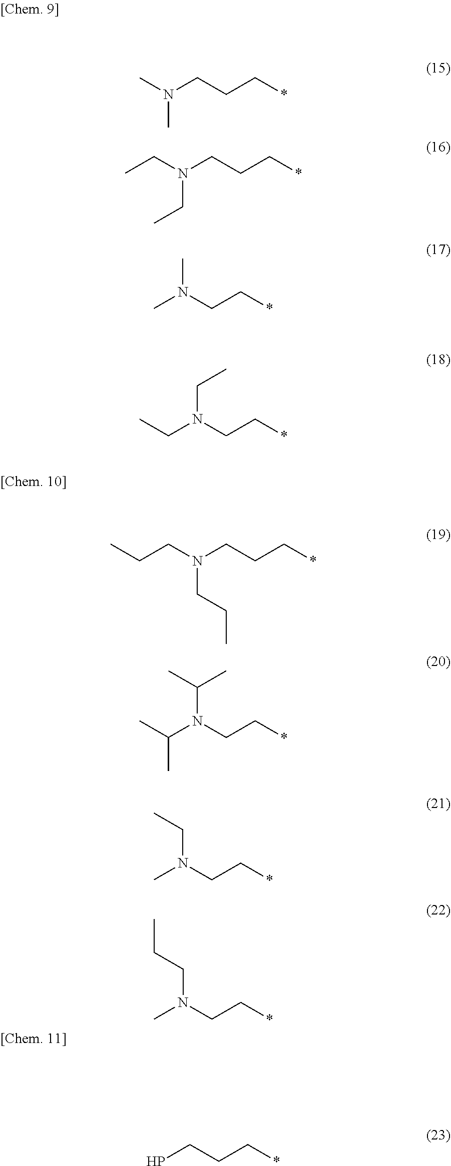

In another preferred embodiment of the present invention, R¹ is group (3), group (5), group (11), group (16), or group (23).

In another preferred embodiment of the present invention, R¹ is group (3), group (5), or group (16).

In the formula (1), R^{2a} and R^{2b} are each independently an alkylene group having 1 to 16 carbon atoms and containing at least one degradable bond. In the present specification, the number of carbon atoms in the “alkylene group” does not include the number of carbon atoms in a degradable bond (e.g., ester bond) that the “alkylene group” may contain. R^{2a} and R^{2b} may be the same or different. R^{2a} and R^{2b} are preferably the same groups.

In the present specification, the alkylene group may be linear or branched. Examples of the alkylene group include methylene group, ethylene group, trimethylene group (—(CH₂)₃—), propylene group (—CH(CH₃)CH₂—, —CH₂CH(CH₃)—), tetramethylene group (—(CH₂)₄—), butylene group (—CH(C₂H₅) CH₂—, —CH₂CH(C₂H₅)—), pentamethylene group (—(CH₂)₅—), hexamethylene group (—(CH₂)₆—), heptamethylene group (—(CH₂)₇—), octamethylene group (—(CH₂)—), nonamethylene group (—(CH₂)₉—), decamethylene group (—(CH₂)₁₀—), undecamethylene group (—(CH₂)—), dodecamethylene group (—(CH₂)₁₂—), tridecamethylene group (—(CH₂)₁₃—), tetradecamethylene group (—(CH₂)₁₄—), pentadecamethylene group (—(CH₂)₁₅—), and hexadecamethylene group (—(CH₂)₁₆—) (in the aforementioned formulas, “ [symbol] ” is a single bond).

In the present specification, the “degradable bond” means a bond that can be hydrolyzed by an intracellular enzyme. Examples of the degradable bond include ester bond, carbamate bond, and disulfide bond.

In the present specification, the “ester bond” means —CO—O— or —O—CO— (in the aforementioned formulas, “ [symbol] ” is a single bond).

In the present specification, the “carbamate bond” means —O—CO—NH— or —NH—CO—O— (in the aforementioned formulas, “ [symbol] ” is a single bond).

In the present specification, the “disulfide bond” means —S—S— (“ [symbol] ” in the aforementioned formula is a single bond).

In one embodiment of the present invention, R^{2a} and R^{2b} are preferably each independently an alkylene group having 1 to 16 carbon atoms and containing at least one degradable bond selected from the group consisting of an ester bond, a carbamate bond, and a disulfide bond.

In another embodiment of the present invention, R^{2a} and R^{2b} are preferably each independently a divalent group represented by the formula (2):

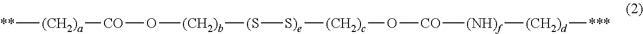

wherein ** is a bonding position to the nitrogen atom in the formula (1),
*** is a bonding position to the carbon atom of the benzene ring in the formula (1),
a to d are each independently an integer of 1 to 4, and
e and f are each independently 0 or 1.

In the formula (2), a is preferably 2 or 3, more preferably 2.

In the formula (2), b and c are each independently preferably an integer of 1 to 3, more preferably 2 or 3.

In the formula (2), the total of b and c is preferably an integer of 2 to 6, more preferably an integer of 4 to 6.

In the formula (2), d is preferably 1 or 2, more preferably 1.

In the formula (2), “e is 0” means that “(S—S)ₑ—” is not present in the formula (2). That is, a divalent group (2) wherein e is 0 is a divalent group represented by the formula (2e):

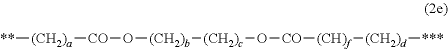

wherein the definitions of the symbols in the formula are as described in the present specification.

In the formula (2), “f is 0” means that “(NH)_{f}—” is not present in the formula (2). That is, a divalent group (2) wherein f is 0 is a divalent group represented by the formula (2f):

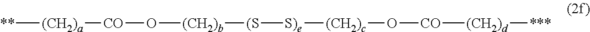

wherein the definitions of the symbols in the formula are as described in the present specification. f is preferably 0.

In the formula (2), a preferable combination of a to f is as follows: a is 2 or 3, b is an integer of 1 to 3, c is an integer of 1 to 3, the total of b and c is an integer of 2 to 6, d is 1 or 2, and e and f are each independently 0 or 1.

In the formula (2), a more preferable combination of a to f is as follows: a is 2, b is 2 or 3, c is 2 or 3, the total of b and c is preferably an integer of 4 to 6, d is 1, e is 0 or 1, and f is 0.

In the formula (1), R^{3a} and R^{3b} are each independently an alkoxy group having 1 to 4 carbon atoms or a halogen atom, and ma and mb are each independently an integer of 0 to 4. R^{3a} and R^{3b} may be the same or different. R^{3a} and R^{3b} are preferably the same groups.

In the present specification, the alkoxy group may be linear or a branched chain. Examples of the alkoxy group include methoxy group, ethoxy group, propoxy group, isopropoxy group, and tert-butoxy group.

In the present specification, examples of the halogen atom include fluorine atom, chlorine atom, bromine atom, and iodine atom.

ma and mb are each the number of R^{3a} and R^{3b}, which are substituents on the benzene ring. In the formula (1), “ma is 0” means that R^{3a} is not present in the formula (1). In the formula (1), “mb is 0” means that R^{3b} is not present in the formula (1). ma and mb are preferably both 0.

In the formula (1), R^{4a} and R^{4b} are each independently R⁵—CO—O—* (wherein * is a bonding position), and R⁵ is a monovalent aliphatic hydrocarbon group having 3 to 30 carbon atoms and optionally containing at least one degradable bond (hereinafter sometimes referred to as “aliphatic hydrocarbon group for R⁵”), or R⁶—CO—(CH₂)ₚ—* (wherein * is a bonding position, R⁶ is a residue of a liposoluble vitamin having a hydroxyl group or a residue of a sterol derivative having a hydroxyl group, and p is an integer of 1 to 8). R^{4a} and R^{4b} may be the same or different. R^{4a} and R^{4b} are preferably the same groups.

In the present specification, examples of the monovalent aliphatic hydrocarbon group include alkyl group, alkenyl group, and alkynyl group. The monovalent aliphatic hydrocarbon group is preferably linear or a branched chain. In the present specification, the number of carbon atoms in the “monovalent aliphatic hydrocarbon group” does not include the number of carbon atoms in a degradable bond (e.g., ester bond) that the “monovalent aliphatic hydrocarbon group” may contain. The number of carbon atoms in the “alkyl group”, the number of carbon atoms in the “alkenyl group”, and the number of carbon atoms in the “alkynyl group” are the same as the number of carbon atoms in the “monovalent aliphatic hydrocarbon group”.

In the present specification, the alkyl group may be linear or branched. Examples of the alkyl group include methyl group, ethyl group, propyl group, isopropyl group, butyl group, isobutyl group, sec-butyl group, tert-butyl group, pentyl group, isopentyl group, neopentyl group, tert-pentyl group, hexyl group, heptyl group, octyl group, nonyl group, decyl group, undecyl group, dodecyl group, tridecyl group, tetradecyl group, pentadecyl group, hexadecyl group, heptadecyl group, octadecyl group, nonadecyl group, icosyl group, henicosyl group, docosyl group, tricosyl group, tetracosyl group, pentacosyl group, hexacosyl group, heptacosyl group, octacosyl group, nonacosyl group, triacontyl group, hentriacontyl group, dotriacontyl group, tritriacontyl group, tetratriacontyl group, pentatriacontyl group, hexatriacontyl group, tetracontyl group, hentetracontyl group, dotetracontyl group, tritetracontyl group, and tetratetracontyl group.

In the present specification, the alkenyl group may be linear or branched. In the present specification, the number of the olefinic carbon-carbon double bond in the alkenyl group may be only one, or two or more. Examples of the alkenyl group include ethenyl group, propenyl group, butenyl group, pentenyl group, hexenyl group, heptenyl group, octenyl group, nonenyl group, decenyl group, undecenyl group, dodecenyl group, tridecenyl group, tetradecenyl group, pentadecenyl group, hexadecenyl group, heptadecenyl group, octadecenyl group, nonadecenyl group, icosenyl group, henicosenyl group, docosenyl group, tricosenyl group, tetracosenyl group, pentacosenyl group, hexacosenyl group, heptacosenyl group, octacosenyl group, nonacosenyl group, triacontenyl group, hentriacontenyl group, and dotriacontenyl group.

In the present specification, the alkynyl group may be linear or branched. In the present specification, the number of the carbon-carbon triple bond of the alkynyl group may be only one, or two or more. Examples of the alkynyl group include ethynyl group, propynyl group, butynyl group, pentynyl group, hexynyl group, heptynyl group, octynyl group, nonynyl group, decynyl group, undecynyl group, dodecynyl group, tridecynyl group, tetradecynyl group, pentadecynyl group, hexadecynyl group, heptadecynyl group, octadecynyl group, nonadecynyl group, icosynyl group, henicosynyl group, docosynyl group, tricosynyl group, tetracosinyl group, pentacosinyl group, hexacosinyl group, heptacosinyl group, octacosinyl group, nonacosinyl group, triacontinyl group, hentriacontinyl group, and dotriacontinyl group.

The number of carbon atoms in the aliphatic hydrocarbon group for R⁵ is preferably 3 to 30. When the aforementioned aliphatic hydrocarbon group is an alkyl group optionally containing at least one degradable bond, the number of carbon atoms in the aforementioned alkyl group is preferably 3 to 30. When the aforementioned aliphatic hydrocarbon group is an alkenyl group optionally containing at least one degradable bond, the number of carbon atoms in the aforementioned alkenyl group is preferably 3 to 30, more preferably 10 to 30.

The degradable bond that may be contained in the aliphatic hydrocarbon group for R⁵ is preferably at least one degradable bond selected from the group consisting of a disulfide bond and an ester bond, more preferably a disulfide bond or an ester bond.

In the aforementioned “R⁶—CO—(CH₂)ₚ—*”, R⁶ is a residue of a liposoluble vitamin having a hydroxy group or a residue of a sterol derivative having a hydroxy group, and p is an integer of 1 to 8. The p is preferably 2 or 3.

In the present specification, the “residue of a liposoluble vitamin having a hydroxy group” means a monovalent group having a structure obtained by removing a hydrogen atom from the hydroxy group of the aforementioned liposoluble vitamin. Examples of the liposoluble vitamin having a hydroxy group include retinol, ergosterol, 7-dehydrocholesterol, calciferol, cholecalciferol, dihydroergocalciferol, dihydrotachysterol, tocopherol, tocotrienol, and the like.

In the present specification, the “residue of a sterol derivative having a hydroxy group” refers to a monovalent group having a structure obtained by removing a hydrogen atom from the hydroxy group of the aforementioned sterol derivative. Example of the sterol derivative having a hydroxy group include cholesterol, cholestanol, stigmasterol, β-sitosterol, lanosterol, ergosterol, and the like.

R⁵ is preferably a monovalent aliphatic hydrocarbon group having 3 to 30 carbon atoms and optionally containing at least one degradable bond.

In one preferred embodiment of the present invention,
R⁵ is a monovalent aliphatic hydrocarbon group optionally containing at least one degradable bond selected from the group consisting of a disulfide bond and an ester bond, and
the monovalent aliphatic hydrocarbon group is an alkyl group having 3 to 30 carbon atoms or an alkenyl group having 3 to 30 carbon atoms.

In another preferred embodiment of the present invention, R⁵ is an alkyl group having 3 to 30 carbon atoms and optionally containing a disulfide bond, or an alkenyl group having 3 to 30 carbon atoms and optionally containing an ester bond. Examples of the alkyl group having 3 to 30 carbon atoms and containing a disulfide bond include R⁵ of compound 29 in the Examples. Examples of the alkenyl group having 3 to 30 carbon atoms and containing an ester bond include R⁵ of compound 28 in the Examples.

In another preferred embodiment of the present invention, R⁵ is an alkenyl group having 10 to 30 carbon atoms.

Preferred examples of cationic lipid (1) include the following cationic lipids.

### [Cationic Lipid (1-1)]

Cationic lipid (1-1), wherein
R¹ is an alkyl group having 1 to 4 carbon atoms and having a nitrogen-containing heterocyclic group, a tertiary amino group, or a hydroxy group as a substituent,
the nitrogen-containing heterocyclic group is a pyrrolidinyl group, a morpholinyl group, a piperazinyl group, a piperidyl group, an azepanyl group, a pyrrolyl group, a pyrazolyl group, a pyridyl group, or an indolyl group,
the tertiary amino group is a di(alkyl)amino group, and
the carbon numbers of the two alkyl groups in the di(alkyl)amino group are each independently 1 to 4,
R^{2a} and R^{2b} are each independently a divalent group represented by the formula (2):

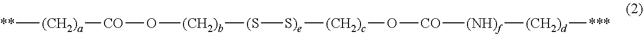

wherein ** is a bonding position to the nitrogen atom in the formula (1),
*** is a bonding position to the carbon atom of the benzene ring in the formula (1),
a to d are each independently an integer of 1 to 4, and
e and f are each independently 0 or 1,
ma and mb are both 0,
R^{4a} and R^{4b} are each independently R⁵—CO—O—* (wherein * is a bonding position), and
R⁵ is a monovalent aliphatic hydrocarbon group optionally containing at least one degradable bond selected from the group consisting of a disulfide bond and an ester bond, and
the monovalent aliphatic hydrocarbon group is an alkyl group having 3 to 30 carbon atoms or an alkenyl group having 3 to 30 carbon atoms.

### [Cationic Lipid (1-2)]

Cationic lipid (1-2), wherein
R¹ is an alkyl group having 1 to 4 carbon atoms and having a nitrogen-containing heterocyclic group, a tertiary amino group, or a hydroxy group as a substituent,
the nitrogen-containing heterocyclic group is a pyrrolidinyl group, a morpholinyl group, or an indolyl group,
the tertiary amino group is a di(alkyl)amino group, and
the carbon numbers of the two alkyl groups in the di(alkyl)amino group are each independently 1 to 4,
R^{2a} and R^{2b} are each independently a divalent group represented by the formula (2):

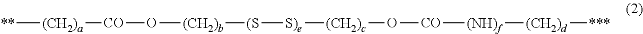

wherein ** is a bonding position to the nitrogen atom in the formula (1),
*** is a bonding position to the carbon atom of the benzene ring in the formula (1),
a is 2 or 3,
b is an integer of 1 to 3,
c is an integer of 1 to 3, the total of b and c is an integer of 2 to 6,
d is 1 or 2, and
e and f are each independently 0 or 1,
ma and mb are both 0,
R^{4a} and R^{4b} are each independently R⁵—CO—O—* (wherein * is a bonding position), and
R⁵ is an alkyl group having 3 to 30 carbon atoms and optionally containing a disulfide bond, or an alkenyl group having 3 to 30 carbon atoms and optionally containing an ester bond.

### [Cationic Lipid (1-3)]

Cationic lipid (1-3), wherein
R¹ is an alkyl group having 1 to 4 carbon atoms and having a nitrogen-containing heterocyclic group or a tertiary amino group as a substituent,
the nitrogen-containing heterocyclic group is a pyrrolidinyl group or a morpholinyl group,
the tertiary amino group is a di(alkyl)amino group, and
the carbon numbers of the two alkyl groups in the di(alkyl)amino group are each independently 1 to 4,
R^{2a} and R^{2b} are each independently a divalent group represented by the formula (2):

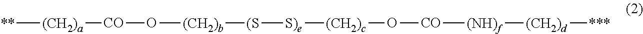

wherein ** is a bonding position to the nitrogen atom in the formula (1),
*** is a bonding position to the carbon atom of the benzene ring in the formula (1),
a is 2,
b is 2 or 3,
c is 2 or 3,
the total of b and c is an integer of 4 to 6,
d is 1,
e is 0 or 1, and
f is 0,
ma and mb are both 0,
R^{4a} and R^{4b} are each independently R⁵—CO—O—* (wherein * is a bonding position), and
R⁵ is an alkenyl group having 3 to 30 carbon atoms.

Specific examples of cationic lipid (1) include compound 1 to compound 30 synthesized in the below-mentioned Examples.

In one embodiment of the present invention, cationic lipid (1) is
preferably at least one selected from the group consisting of compound 1 to compound 30,
more preferably at least one selected from the group consisting of compound 1, compound 3, compound 6, compound 7, and compound 27,
further preferably at least one selected from the group consisting of compound 3, compound 7, and compound 27.

The cationic lipid (1) of the present invention can be synthesized using commercially available starting materials and in the same manner as in the Examples described below. For example, a cationic lipid (1) in which R^{2a} and R^{2b} are both divalent groups represented by the formula (2), e in the formula (2) is 1, and f in the formula (2) is 0 can be synthesized using commercially available starting materials and in the same manner as in the below-mentioned Example 1 or Example 2. For example, a cationic lipid (1) in which R^{2a} and R^{2b} are both divalent groups represented by the formula (2), and e and f in the formula (2) are both 1 can be synthesized using commercially available starting materials and in the same manner as in the below-mentioned Example 22. For example, a cationic lipid (1) in which R^{2a} and R^{2b} are both divalent groups represented by the formula (2), and e and f in the formula (2) are both 0 can be synthesized using commercially available starting materials and in the same manner as in the below-mentioned Example 24. The conditions for the synthetic reaction (e.g., reaction temperature, reaction time, etc.) can be appropriately determined by those of ordinary skill in the art.

The present invention also provides a lipid membrane structure containing cationic lipid (1) as a constituent lipid of the membrane. The lipid membrane structure of the present invention may further contain a nucleic acid.

In the present specification, the “lipid membrane structure” means a particle having a membrane structure in which the hydrophilic groups of amphipathic lipid are arranged in the interface, facing the aqueous phase side. The “amphiphilic lipid” means a lipid having both a hydrophilic group showing hydrophilicity and a hydrophobic group showing hydrophobicity. Examples of the amphiphilic lipid include cationic lipid, phospholipid, and the like.

While the form of the lipid membrane structure of the present invention is not particularly limited, for example, liposome (e.g., monolayer liposome, multilayer liposome, and the like), O/W emulsion, W/O emulsion, spherical micelle, worm-like micelle, lipid nano particle (sometimes to be abbreviated as “LNP” in the present specification), disordered layer structure, and the like can be mentioned as a form of cationic lipid (1) dispersed in an aqueous solvent. The lipid membrane structure of the present invention is preferably a liposome or LNP, more preferably LNP.

The lipid membrane structure of the present invention may further contain other constituent components in addition to cationic lipid (1). Examples of said other constituent component include lipid (phospholipid (phosphatidylinositol, phosphatidylethanolamine, phosphatidylserine, phosphatidic acid, phosphatidylglycerol, phosphatidylcholine, etc.), glycolipid, peptide lipid, cholesterol, cationic lipid other than cationic lipid (1), PEG lipid, etc.), surfactant (e.g., 3-[(3-cholamidopropyl)dimethylammonio]propanesulfonate, sodium cholate salt, octylglycoside, N-D-gluco-N-methylalkane amides, etc.), polyethylene glycol, protein, and the like. The content of said other constituent component in the lipid membrane structure of the present invention is, for example, 5 to 95 mol %, preferably to 90 mol %, more preferably 30 to 80 mol %, relative to all constituent components in the lipid membrane structure of the present invention.

While the content of cationic lipid (1) to be contained in the lipid membrane structure of the present invention is not particularly limited, when the lipid membrane structure is used as the below-mentioned nucleic acid-introducing agent, cationic lipid (1) is contained in an amount sufficient for introducing a nucleic acid. The content of cationic lipid (1) in the lipid membrane structure of the present invention is, for example, 5 to 95 mol %, preferably 10 to 90 mol %, more preferably 20 to 70 mol %, relative to all constituent components in the lipid membrane structure of the present invention.

The lipid membrane structure of the present invention can be prepared by dispersing cationic lipid (1) and other constituent components (lipid etc.) in a suitable solvent or dispersion medium, for example, aqueous solvent and alcoholic solvent, and performing an operation to induce organization as necessary.

Examples of the “operation to induce organization” include, but are not limited to, methods known per se such as ethanol dilution method using a micro flow path or vortex, simple hydration method, sonication, heating, vortex, ether injecting method, French press method, cholic acid method, Ca²⁺ fusion method, freeze-thaw method, reversed-phase evaporation method, and the like.

A nucleic acid can be introduced into a cell in vivo and/or in vitro by bringing a lipid membrane structure containing the nucleic acid into contact with the cell. Therefore, the present invention provides a nucleic acid-introducing agent containing cationic lipid (1). The nucleic acid-introducing agent of the present invention is preferably the lipid structure of the present invention. The nucleic acid-introducing agent of the present invention preferably contains a nucleic acid.

The nucleic acid-introducing agent of the present invention can introduce any nucleic acid into a cell. The type of nucleic acid includes, but is not limited to, DNA, RNA, RNA chimeric nucleic acid, DNA/RNA hybrid, and the like. While any of single-stranded to triple-stranded nucleic acids can be used, single-stranded or double-strand one is preferred. The nucleic acid may also be other type of nucleotide that is N-glycoside of a purine or pyrimidine base, other oligomer having a non-nucleotide backbone (e.g., commercially available peptide nucleic acid (PNA) and the like), other oligomer containing a special bond (said oligomer containing a nucleotide having a configuration permitting base pairing or base attachment, which are found in DNA and RNA), or the like. Furthermore, the nucleic acid may also be, for example, a nucleic acid added with known modification, a nucleic acid with a label known in the pertinent field, a nucleic acid with a cap, a methylated nucleic acid, a nucleic acid in which one or more natural nucleotides are substituted by an analog, a nucleic acid with an intramolecularly modified nucleotide, a nucleic acid with a non-charge bond (e.g., methylphosphonate, phosphotriester, phosphoramidate, carbamate, etc.), a nucleic acid with a charged bond or sulfur-containing bond (e.g., phosphorothioate, phosphorodithioate, etc.), a nucleic acid with a side chain group such as protein (nuclease, nuclease inhibitor, toxin, antibody, signal peptide, poly-L-lysine, etc.), sugar (e.g., monosaccharide, etc.), or the like, a nucleic acid containing an intercalating compound (e.g., acridine, psoralen, etc.), a nucleic acid containing a chelate compound (e.g., metal, radioactive metal, boron, oxidative metal, etc.), a nucleic acid containing an alkylating agent, a nucleic acid with a modified bond (e.g., a anomer-type nucleic acid, etc.), or the like.

The type of DNA that can be used in the present invention is not particularly limited and can be selected appropriately depending on the object of use. Examples include plasmid DNA, cDNA, antisense DNA, chromosomal DNA, PAC, BAC, CpG oligos, etc., with plasmid DNA, cDNA, and antisense DNA being preferred, and plasmid DNA being more preferred. Circular DNA such as plasmid DNA can also be digested with appropriate restriction enzymes and used as linear DNA.

The type of RNA that can be used in the present invention is not particularly limited and can be selected appropriately depending on the object of use. Examples include siRNA, miRNA, shRNA, antisense RNA, messenger RNA (mRNA), single-stranded RNA genome, double-stranded RNA genome, RNA replicon, transfer RNA, ribosomal RNA, and the like, and siRNA, miRNA, shRNA, mRNA, antisense RNA, and RNA replicon are preferred.

The nucleic acid used in the present invention is preferably purified by a method generally used by those of ordinary skill in the art.

The nucleic acid-introducing agent of the present invention containing a nucleic acid can be administered in vivo for the purpose of, for example, prevention and/or treatment of diseases. Therefore, the nucleic acid to be used in the present invention is preferably one having a preventive and/or therapeutic activity against a given disease (prophylactic/therapeutic nucleic acid). Examples of such nucleic acid include nucleic acids used for so-called gene therapy, and the like.

In order to introduce a nucleic acid into a cell by using the nucleic acid-introducing agent of the present invention, the lipid membrane structure of the present invention containing the nucleic acid (i.e., the nucleic acid-introducing agent of the present invention containing the nucleic acid) is formed by achieving the co-presence of the nucleic acid of interest when forming the lipid membrane structure of the present invention. For example, when a liposome is formed as the lipid membrane structure by an ethanol dilution method, an aqueous nucleic acid solution and an ethanol solution of the constituent components (lipid and the like) of the lipid membrane structure of the present invention are vigorously mixed in a vortex, micro flow path, or the like, and the mixture is diluted with an appropriate buffer. When a liposome is formed as the lipid membrane structure by a simple hydration method, the constituent components (lipid and the like) of the lipid membrane structure of the present invention are dissolved in an appropriate organic solvent, and the solution is placed in a glass container and dried under reduced pressure to evaporate the solvent, whereby a lipid thin film is obtained. An aqueous solution of nucleic acid is added thereto, and after hydration, the mixture is subjected to ultrasonic treatment using a sonicator.

One form of the lipid membrane structure of the present invention containing a nucleic acid is LNP encapsulating the nucleic acid by forming an electrostatic complex between the nucleic acid and a cationic lipid. This LNP can be used as a drug delivery system for selectively delivering a nucleic acid and the like into a particular cell, and is useful for, for example, DNA vaccines by introduction of antigen gene into dendritic cells, gene therapy drugs for tumor, nucleic acid pharmaceutical products that suppress expression of target genes by utilizing RNA interference, and the like.

The particle size of the lipid membrane structure of the present invention containing a nucleic acid is not particularly limited, and is preferably 10 nm to 500 nm, more preferably 30 nm to 200 nm. The particle size can be measured by using a particle size distribution measuring device such as Zetasizer Nano (Malvern) and the like. The particle size of the lipid membrane structure can be appropriately adjusted by the method for preparing the lipid membrane structure.

The surface potential (zeta potential) of the lipid membrane structure of the present invention containing a nucleic acid is not particularly limited and preferably −15 mV to +15 mV, more preferably −10 mV to +10 mV. In conventional gene transfer, particles electrically charged to have a plus surface potential have been mainly used. This is useful as a method for promoting electrostatic interactions with negatively-charged heparin sulfate on the cell surface to enhance uptake into cells. However, the positive surface potential may suppress release of nucleic acid from the carrier in the cell due to the interaction with a nucleic acid to be delivered, or protein synthesis due to the interaction between mRNA and a nucleic acid to be delivered. This problem can be solved by adjusting the surface potential to fall within the above-mentioned range. The surface potential can be measured using a zeta potential measuring device such as Zetasizer Nano and the like. The surface potential of the lipid membrane structure can be adjusted by the composition of the constituent component of the lipid membrane structure containing cationic lipid (1).

The lipid membrane surface pKa (hereinafter sometimes to be abbreviated as “Liposomal pKa”) of the lipid membrane structure of the present invention is not particularly limited, and is preferably 4.5 to 9.5. Liposomal pKa is used as an index indicating the susceptibility of protonation of a lipid membrane structure which is incorporated by endocytosis in an endosome in a weakly acidic environment inside the endosome. As described in Angewante Chemie International Edition 51: 8529-8533, 2012 or Molecular Therapy 24(4): 786-795, 2016, to escape from an endosome and deliver a nucleic acid into a cytoplasm, it is important to set the Liposomal pKa to a value preferable for the escape from the endosome. By adjusting the Liposomal pKa to fall within the above ranges, a nucleic acid can be efficiently delivered into a cytoplasm. The Liposomal pKa can be adjusted by the composition of the constituent component of a lipid membrane structure containing cationic lipid (1).

By contacting a cell with the nucleic acid-introducing agent of the present invention containing a nucleic acid (preferably the lipid membrane structure of the present invention containing nucleic acid), the nucleic acid contained in the aforementioned nucleic acid-introducing agent can be introduced into the aforementioned cell. Therefore, the present invention also provides a method for introducing the nucleic acid contained in the nucleic acid-introducing agent into the cell, which includes contacting the cell with the nucleic acid-introducing agent of the present invention containing the nucleic acid.

The kind of the aforementioned cell is not particularly limited, and cells of prokaryote and eukaryote can be used. Preferred is eucaryote. The kind of the eucaryote is also not particularly limited, and examples thereof include vertebrates such as mammals including human (e.g., human, monkey, mouse, rat, hamster, bovine, etc.), birds (e.g., chicken, ostrich, etc.), amphibia (e.g., frog, etc.), fishes (e.g., zebrafish, rice-fish, etc.) and the like, invertebrates such as insects (silk moth, moth, Drosophila, etc.), and the like, plants, microorganisms (e.g., yeasts), and the like. The cell to be the target in the present invention is more preferably an animal or plant cell, further preferably a mammalian cell. The cell may be a culture cell line including a cancer cell, or a cell isolated from an individual or tissue, or a cell of a tissue or tissue piece. The cell may be an adherent cell or a non-adherent cell.

The step of contacting the lipid membrane structure of the present invention encapsulating a nucleic acid with a cell in vitro is specifically described below.

Cells are suspended in a suitable medium several days before contact with the lipid membrane structure, and cultured under appropriate conditions. At the time of contact with the lipid membrane structure, the cells may or may not be in a proliferative phase.

The culture medium at the time of the contact may be a serum-containing medium or a serum-free medium. The serum concentration of the medium is preferably not more than 30 wt %, more preferably not more than 20 wt %. When the medium contains excess proteins such as serum and the like, the contact between the lipid membrane structure and the cell may be inhibited.

The cell density at the time of the contact is not particularly limited, and can be appropriately determined in consideration of the kind of the cell and the like. It is generally within the range of 1×10⁴ to 1×10⁷ cells/mL.

For example, a suspension of the lipid membrane structure of the present invention containing a nucleic acid (i.e., the nucleic acid-introducing agent of the present invention containing a nucleic acid) is added to cells. The amount of the suspension to be added is not particularly limited, and can be appropriately determined in consideration of the cell number and the like. The concentration of the lipid membrane structure when contacting cells is not particularly limited as long as the desired introduction of the nucleic acid into the cells can be achieved. The lipid concentration is generally 1 to 100 nmol/ml, preferably 10 to 50 nmol/ml, and the nucleic acid concentration is generally 0.01 to 100 μg/ml, preferably 0.1 to 10 μg/ml.

After the aforementioned suspension is added to cells, the cells are cultured. The temperature, humidity, CO₂ concentration, and the like during culturing are appropriately determined in consideration of the kind of the cell. When the cell is derived from a mammal, generally, the temperature is about 37° C., the humidity is about 95%, and the CO₂ concentration is about 5 vol %. While the culture time can also be appropriately determined in consideration of the conditions such as the kind of the cell and the like, it is generally a range of 0.1 to 76 hr, preferably a range of 0.2 to 24 hr, more preferably a range of 0.5 to 12 hr. When the above-mentioned culture time is too short, the nucleic acid is not sufficiently introduced into the cells, and when the culture time is too long, the cells may become weak.

By the above-mentioned culture, a nucleic acid is introduced into cells. The culture is further continued preferably by exchanging the medium with a fresh medium, or adding a fresh medium to the medium. When the cell is a mammal-derived cell, the fresh medium preferably contains a serum or nutrition factor.

As mentioned above, a nucleic acid can be introduced into cells not only in vitro but also in vivo by using the nucleic acid-introducing agent of the present invention containing a nucleic acid. That is, by administration of the nucleic acid-introducing agent containing a nucleic acid to a living organism, the nucleic acid contained in the nucleic acid-introducing agent can be introduced into the target cells in the aforementioned living organism. Therefore, the present invention also provides a method for introducing a nucleic acid contained in a nucleic acid-introducing agent into a target cell in a living organism, comprising administering the nucleic acid-introducing agent of the present invention containing the nucleic acid to the living organism.

The living organisms to which the nucleic acid-introducing agent of the present invention containing a nucleic acid can be administered are not particularly limited, and examples thereof include vertebrates such as mammals (e.g., human, monkey, mouse, rat, hamster, bovine, etc.), birds (e.g., chicken, ostrich, etc.), amphibia (e.g., frog, etc.), fishes (e.g., zebrafish, rice-fish, etc.), and the like, invertebrates such as insects (e.g., silk moth, moth, Drosophila, etc.) and the like, plants, and the like. The living organisms to which the nucleic acid-introducing agent of the present invention containing a nucleic acid can be administered are preferably humans or other mammals.

The kind of the target cell is not particularly limited, and a nucleic acid can be introduced into cells in various tissues (e.g., liver, kidney, pancreas, lung, spleen, heart, blood, muscle, bone, brain, stomach, small intestine, large intestine, skin, adipose tissue, lymph node, tumor, etc.) by using the nucleic acid-introducing agent of the present invention containing a nucleic acid.

The method of administering the nucleic acid-introducing agent of the present invention containing a nucleic acid to a living organism is not particularly limited, and an administration method known per se (e.g., oral administration, parenteral administration (e.g., intravenous administration, intramuscular administration, topical administration, transdermal administration, subcutaneous administration, intraperitoneal administration, spray, etc.), etc.) can be appropriately selected. The dose of the nucleic acid-introducing agent of the present invention containing a nucleic acid is not particularly limited, and can be appropriately selected taking into account the kind of living organism to be the subject of administration, the administration method, the type and site of target cells, and the like.

When cationic lipid (1) or lipid membrane structure is used as the nucleic acid-introducing agent, it can be formulated according to a conventional method.

When the nucleic acid-introducing agent of the present invention, which is the lipid membrane structure of the present invention, is provided as a reagent for studies, the lipid membrane structure of the present invention can be provided as is, or as a sterile solution or suspension of the lipid membrane structure of the present invention in, for example, water or another physiologically acceptable liquid (e.g., water-soluble solvent (e.g., malic acid buffer, etc.), an organic solvent (e.g., ethanol, methanol, DMSO, tert-butanol, etc.), or a mixture of a water-soluble solvent and an organic solvent, etc.). The nucleic acid-introducing agent of the present invention may appropriately contain physiologically acceptable additives (e.g., excipient, vehicle, preservative, stabilizer, binder, etc.), which are known per se.

Furthermore, when the nucleic acid-introducing agent of the present invention, which is the lipid membrane structure of the present invention, is provided as a medicament, the lipid membrane structure of the present invention can be used as is, or the lipid membrane structure of the present invention can be used together with known pharmaceutically acceptable additives (e.g., carrier, flavor, excipient, vehicle, preservative, stabilizer, binder, etc.) and mixed in a unit dosage form required for generally accepted pharmaceutical practice, to produce the nucleic acid-introducing agent of the present invention as an oral agent (e.g., tablet, capsule, etc.) or parenteral agent (e.g., injectable preparation, spray, etc.), preferably parenteral agent (more preferably, injectable preparation).

The nucleic acid-introducing agent of the present invention can also be provided in the form of a kit. The kit can contain, in addition to cationic lipid (1) or the lipid membrane structure of the present invention, a reagent used for the introduction of a nucleic acid. In one embodiment, the nucleic acid-introducing agent (or kit) of the present invention further contains a polycation (e.g., protamine). Using the nucleic acid-introducing agent (or kit) of the present invention in this embodiment, an electrostatic complex between a nucleic acid and a polycation (e.g., protamine) can be encapsulated easily in the lipid membrane structure of the present invention, whereby the nucleic acid can be introduced into cells.

The present invention also provides a pharmaceutical composition containing cationic lipid (1). The pharmaceutical composition of the present invention may further contain a nucleic acid. The pharmaceutical composition of the present invention may also contain known pharmaceutically acceptable additives (e.g., carrier, flavor, excipient, vehicle, preservative, stabilizer, binder, etc.).

The pharmaceutical composition of the present invention may be a powder composition obtained by removing the solvent by lyophilization or the like, or a liquid composition. A powder composition may be produced by removing the solvent from a liquid composition by filtration, centrifugation, or the like, or by lyophilizing a liquid composition.

The pharmaceutical composition of the present invention can be formulated as an oral preparation (e.g., tablet, capsule, etc.) or a parenteral preparation (e.g., injectable preparation, spray, etc.), preferably a parenteral preparation (more preferably an injectable preparation). The pharmaceutical composition of the present invention can be formulated not only for adults but also for children.

The present invention also provides a method for producing a cellular medicine containing cells expressing a gene in a nucleic acid, the method including contacting cells with the nucleic acid-introducing agent of the present invention containing the nucleic acid and introducing the nucleic acid contained in the aforementioned nucleic acid-introducing agent into the cell.

The aforementioned cell expressing the gene in the nucleic acid refers to a cell in which a gene of interest has been expressed by introducing the nucleic acid into the cell.

The nucleic acid-introducing agent of the present invention containing a nucleic acid in vitro is brought into contact with cells to introduce the aforementioned nucleic acid into the cells.

Examples of the cell to be used in the production of cellular medicines include T cell, B cell, NK cell, dendritic cell, macrophage, monocyte, and the like. T cells to be used in the production of cellular medicines may be T cells induced to differentiate from lymphocyte precursor cells, including pluripotent cells. Examples of the lymphocyte precursor cell, including pluripotent cell, include embryonic stem cell (ES cell), induced pluripotent stem cell (iPS cell), and the like. Undifferentiated cells, such as pluripotent cell, can be differentiated into T cells by a known method.

Examples of nucleic acid to be used in the production of cellular medicine include nucleic acids encoding chimeric antigen receptors (CARs) and T cell receptors (TCRs).

The nucleic acid encoding CAR to be used in the production of a cellular medicine contains an antigen-binding domain of an antibody capable of specifically recognizing the surface antigen to be recognized by the target immune cell, an extracellular hinge domain, a transmembrane domain, and an intracellular T cell signaling domain.

The nucleic acid encoding TCR to be used in the production of cellular medicines encodes the a chain and R chain of TCR capable of specifically recognizing the surface antigen to be recognized by the target T cell.

The nucleic acid encoding CAR or TCR is not particularly limited, and examples thereof include DNA, RNA, chimera nucleic acid of RNA, DNA/RNA hybrid, and the like.

Cellular medicine contains a cell expressing a specific gene and may further contain pharmaceutically acceptable additives (e.g., carrier, excipient, vehicle, preservative, stabilizer, etc.). Cellular medicine is preferably a parenteral preparation, more preferably an injectable preparation.

The cellular medicine can be used for the treatment or prophylaxis of diseases such as cancer and the like. The cancer to be the application target of the cellular medicine is not particularly limited, and examples thereof include lung cancer, breast cancer, gastric cancer, colon cancer, uterine cancer, ovarian cancer, osteosarcoma, chondrosarcoma, rhabdomyosarcoma, leiomyosarcoma, fibrosarcoma, liposarcoma, angiosarcoma, leukemia, malignant lymphoma, myeloma, and the like.

The subject to which the cellular medicine can be administered is not particularly limited, and examples thereof include mammals (e.g., human, monkey, mouse, rat, hamster, bovine, etc.) and the like. The subject of administration of the cellular medicine is preferably human or other mammals.

The method of administering a cellular medicine is not particularly limited as long as it allows the cells to express the target gene, and parenteral administration (e.g., intravenous administration, intramuscular administration, topical administration, transdermal administration, subcutaneous administration, intraperitoneal administration, spray, and the like), and the like can be appropriately selected in consideration of the kind of the cell, target disease, and the like. The dose of the cellular medicine is not particularly limited as long as the cell can express the target gene, and can be appropriately selected in consideration of the kind of the subject of administration, the administration method, the kind of the cell, target disease, and the like.

Other features of the invention will become apparent in the course of the following descriptions of exemplary embodiments which are given for illustration of the invention and are not intended to be limiting thereof.

### EXAMPLES

The present invention is explained in more detail in the following by referring to Examples and Experimental Examples, but the present invention is not limited by these.

The abbreviations used in the description of Examples each mean the following.
Chol: cholesterol
DMAP: 4-dimethylaminopyridine
DMF: N,N-dimethylformamide
DMG-PEG2k: 1,2-dimyristoyl-rac-glycero-3-methoxypolyethylene glycol (number average molecular weight of PEG chain: 2000)
DMSO: dimethyl sulfoxide
DOPC: 1,2-dioleoyl-sn-glycero-3-phosphocholine
DSC: N,N′-disuccinimidyl carbonate
EDC hydrochloride: 1-ethyl-3-(3-dimethylaminopropyl)carbodiimide hydrochloride
LNP: lipid nano particle
MeOH: methanol
MES: 2-morpholinoethanesulfonic acid
mRNA: messenger RNA
PBS: phosphate buffered saline
TBS: tert-butyldimethylsilyl
tBuOH: tert-butanol
TMS-Cl: trimethylsilyl chloride
TNS: sodium 6-(p-toluidino)-2-naphthalenesulfonate

The structures of compound 1 to compound 30 synthesized in the below-mentioned Example 1 to Example 30 are shown in Table 1-1 to Table 1-5 below, and the structures of the comparison compounds are shown in Table 2 below. Reagents purchased from FUJIFILM Wako Pure Chemical Corporation were used as the comparison compounds.

TABLE 1-1

| name | structure |
|---|---|
| com- pound 1 | 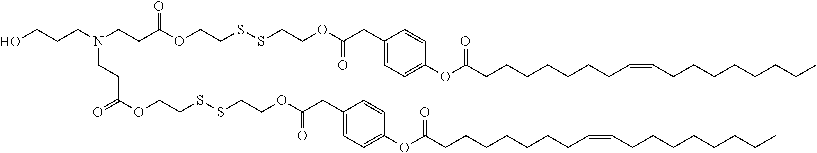 [SMILES: CCCCCCCC/C=C\CCCCCCCC(=O)Oc1ccc(CC(=O)OCCSSCCOC(=O)CCN(CCCO)CCC(=O)OCCSSCCOC(=O)Cc2ccc(OC(=O)CCCCCCC/C=C\CCCCCCCC)cc2)cc1] |
| com- pound 2 | 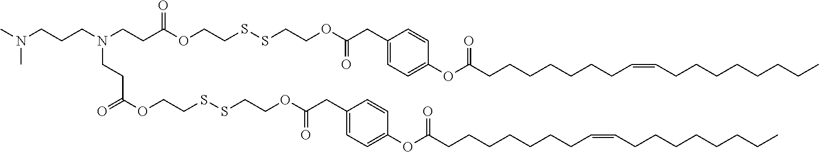 [SMILES: CCCCCCCC/C=C\CCCCCCCC(=O)Oc1ccc(CC(=O)OCCSSCCOC(=O)CCN(CCCN(C)C)CCC(=O)OCCSSCCOC(=O)Cc2ccc(OC(=O)CCCCCCC/C=C\CCCCCCCC)cc2)cc1] |
| com- pound 3 | 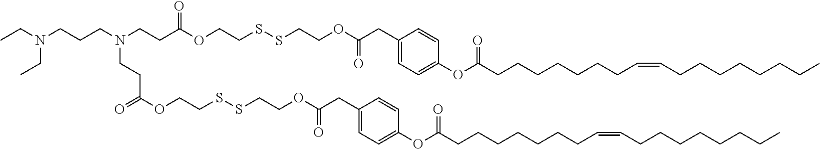 [SMILES: CCCCCCCC/C=C\CCCCCCCC(=O)Oc1ccc(CC(=O)OCCSSCCOC(=O)CCN(CCCN(CC)CC)CCC(=O)OCCSSCCOC(=O)Cc2ccc(OC(=O)CCCCCCC/C=C\CCCCCCCC)cc2)cc1] |
| com- pound 4 | 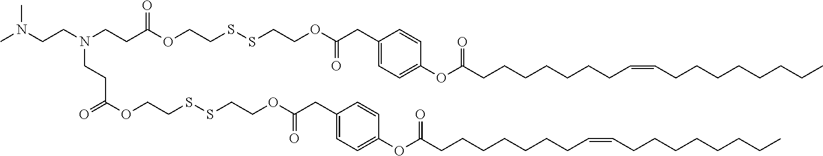 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN(C)C)cc1] |
| com- pound 5 | 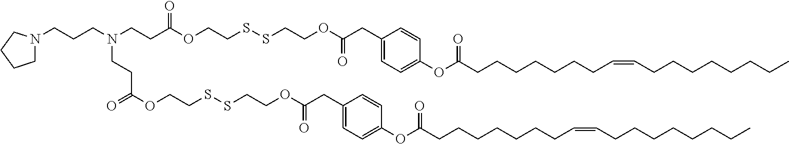 [SMILES: CCCCCCCC/C=C\CCCCCCCC(=O)Oc1ccc(CC(=O)OCCSSCCOC(=O)CCN(CCCN2CCCC2)CCC(=O)OCCSSCCOC(=O)Cc2ccc(OC(=O)CCCCCCC/C=C\CCCCCCCC)cc2)cc1] |
| com- pound 6 | 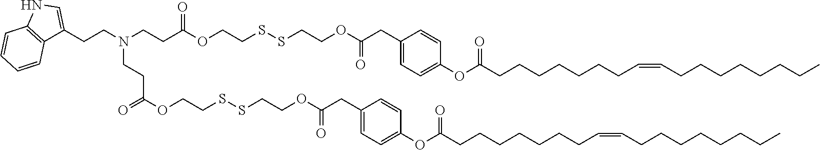 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCc2c[nH]c3ccccc23)cc1] |
| com- pound 7 | 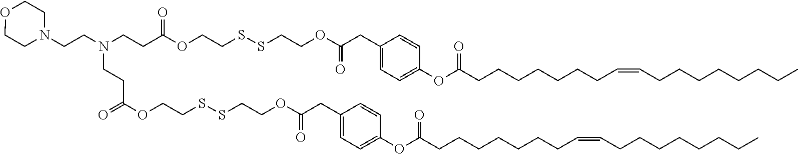 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN2CCOCC2)cc1] |

TABLE 1-2

| name | structure |
|---|---|
| com- pound 8 | 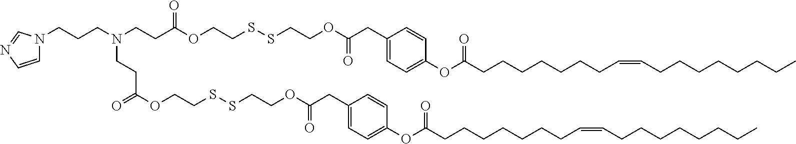 [SMILES: CCCCCCCC/C=C\CCCCCCCC(=O)Oc1ccc(CC(=O)OCCSSCCOC(=O)CCN(CCCn2ccnc2)CCC(=O)OCCSSCCOC(=O)Cc2ccc(OC(=O)CCCCCCC/C=C\CCCCCCCC)cc2)cc1] |
| com- pound 9 | 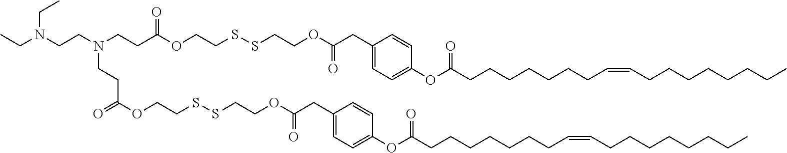 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN(CC)CC)cc1] |
| com- pound 10 | 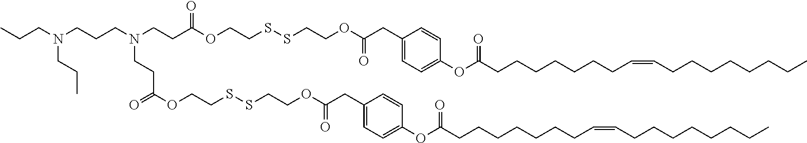 [SMILES: CCCCCCCC/C=C\CCCCCCCC(=O)Oc1ccc(CC(=O)OCCSSCCOC(=O)CCN(CCCN(CCC)CCC)CCC(=O)OCCSSCCOC(=O)Cc2ccc(OC(=O)CCCCCCC/C=C\CCCCCCCC)cc2)cc1] |
| com- pound 11 | 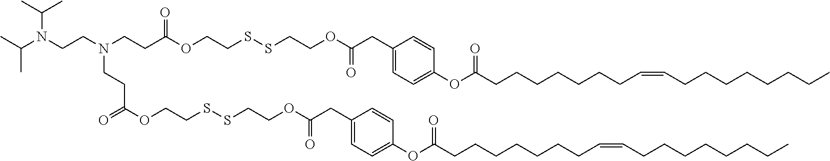 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN(C(C)C)C(C)C)cc1] |
| com- pound 12 | 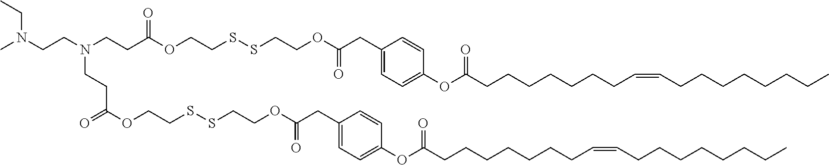 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN(C)CC)cc1] |
| com- pound 13 | 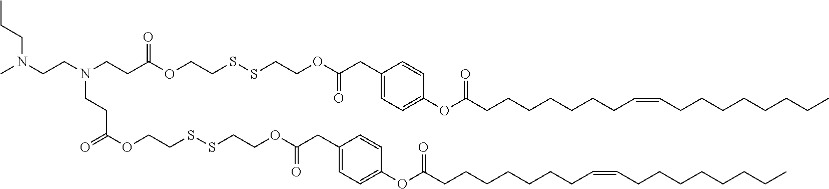 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN(C)CCC)cc1] |
| com- pound 14 | 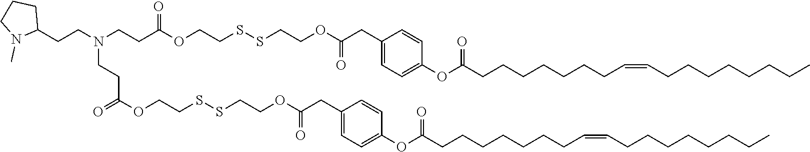 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCC2CCCN2C)cc1] |

TABLE 1-3

| name | structure |
|---|---|
| com- pound 15 | 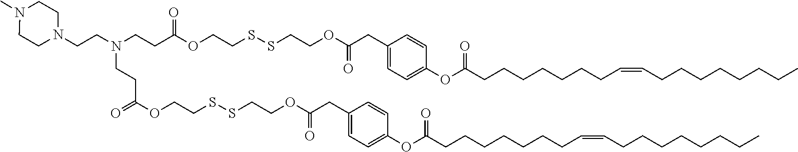 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN2CCN(C)CC2)cc1] |
| com- pound 16 | 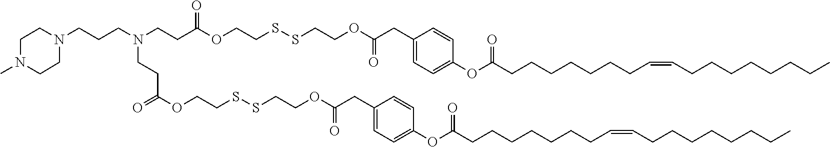 [SMILES: CCCCCCCC/C=C\CCCCCCCC(=O)Oc1ccc(CC(=O)OCCSSCCOC(=O)CCN(CCCN2CCN(C)CC2)CCC(=O)OCCSSCCOC(=O)Cc2ccc(OC(=O)CCCCCCC/C=C\CCCCCCCC)cc2)cc1] |
| com- pound 17 | 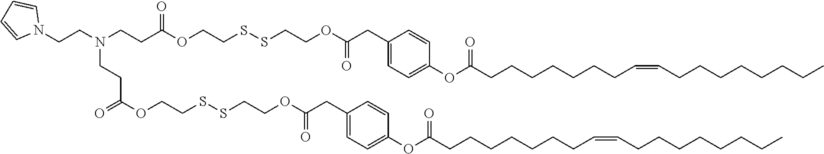 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCn2cccc2)cc1] |
| com- pound 18 | 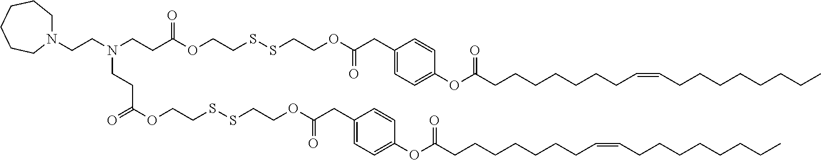 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN2CCCCCC2)cc1] |
| com- pound 19 | 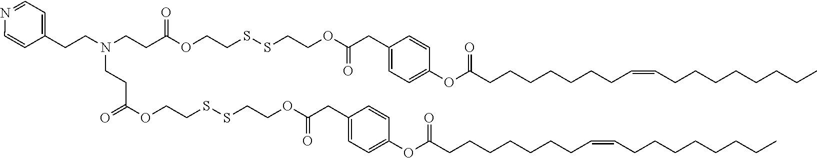 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCc2ccncc2)cc1] |
| com- pound 20 | 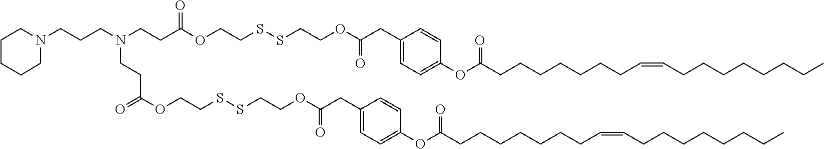 [SMILES: CCCCCCCC/C=C\CCCCCCCC(=O)Oc1ccc(CC(=O)OCCSSCCOC(=O)CCN(CCCN2CCCCC2)CCC(=O)OCCSSCCOC(=O)Cc2ccc(OC(=O)CCCCCCC/C=C\CCCCCCCC)cc2)cc1] |

TABLE 1-4

| name | structure |
|---|---|
| com- pound 21 | 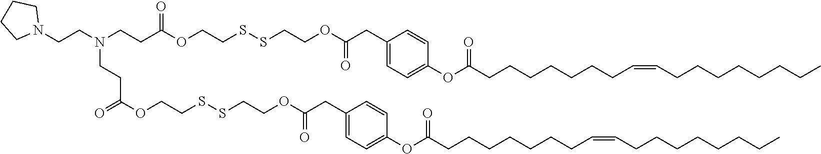 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN2CCCC2)cc1] |
| com- pound 22 | 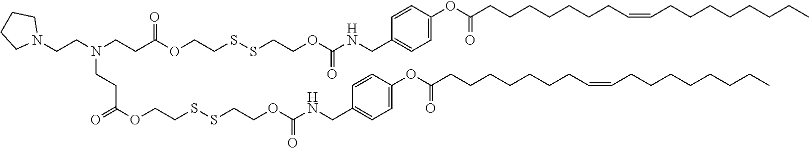 [SMILES: CCCCCCCC/C=C\CCCCCCCC(=O)Oc1ccc(CNC(=O)OCCSSCCOC(=O)CCN(CCC(=O)OCCSSCCOC(=O)NCc2ccc(OC(=O)CCCCCCC/C=C\CCCCCCCC)cc2)CCN2CCCC2)cc1] |
| com- pound 23 | 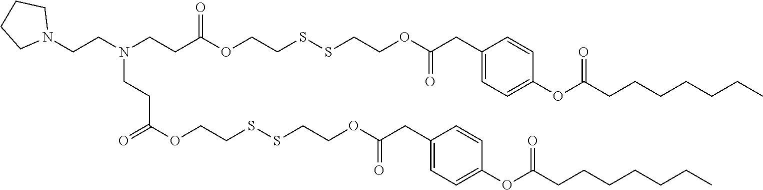 [SMILES: CCCCCCCC(=O)Oc1ccc(CC(=O)OCCSSCCOC(=O)CCN(CCC(=O)OCCSSCCOC(=O)Cc2ccc(OC(=O)CCCCCCC)cc2)CCN2CCCC2)cc1] |
| com- pound 24 | 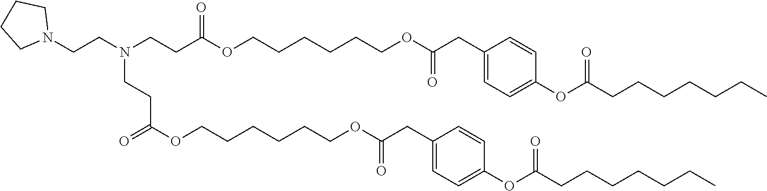 [SMILES: CCCCCCCC(=O)Oc1ccc(CC(=O)OCCCCCCOC(=O)CCN(CCC(=O)OCCCCCCOC(=O)Cc2ccc(OC(=O)CCCCCCC)cc2)CCN2CCCC2)cc1] |
| com- pound 25 | 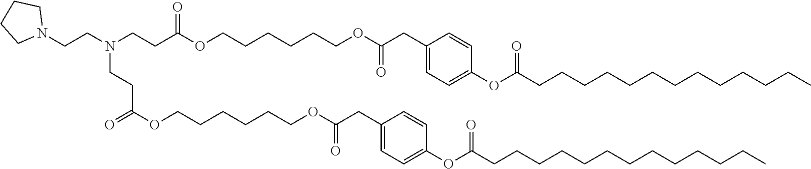 [SMILES: CCCCCCCCCCCCCC(=O)Oc1ccc(CC(=O)OCCCCCCOC(=O)CCN(CCC(=O)OCCCCCCOC(=O)Cc2ccc(OC(=O)CCCCCCCCCCCCC)cc2)CCN2CCCC2)cc1] |
| com- pound 26 | 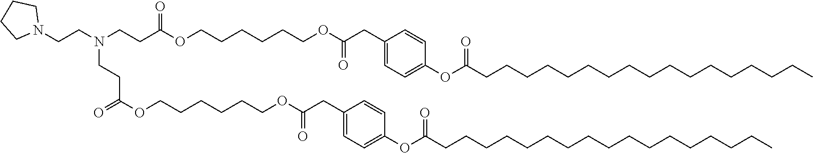 [SMILES: CCCCCCCCCCCCCCCCCC(=O)Oc1ccc(CC(=O)OCCCCCCOC(=O)CCN(CCC(=O)OCCCCCCOC(=O)Cc2ccc(OC(=O)CCCCCCCCCCCCCCCCC)cc2)CCN2CCCC2)cc1] |

TABLE 1-5

| name | structure |
|---|---|
| com- pound 27 | 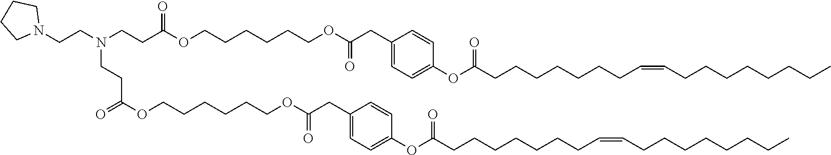 [SMILES: CCCCCCCC/C=C\CCCCCCCC(=O)Oc1ccc(CC(=O)OCCCCCCOC(=O)CCN(CCC(=O)OCCCCCCOC(=O)Cc2ccc(OC(=O)CCCCCCC/C=C\CCCCCCCC)cc2)CCN2CCCC2)cc1] |
| com- pound 28 | 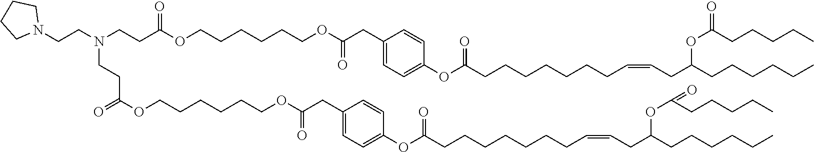 [SMILES: CCCCCCC(C/C=C\CCCCCCCC(=O)Oc1ccc(CC(=O)OCCCCCCOC(=O)CCN(CCC(=O)OCCCCCCOC(=O)Cc2ccc(OC(=O)CCCCCCC/C=C\CC(CCCCCC)OC(=O)CCCCC)cc2)CCN2CCCC2)cc1)OC(=O)CCCCC] |
| com- pound 29 | 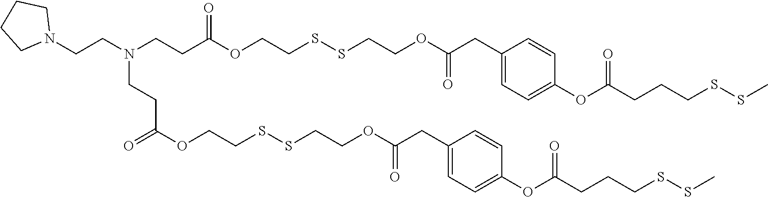 [SMILES: CSSCCCC(=O)Oc1ccc(CC(=O)OCCSSCCOC(=O)CCN(CCC(=O)OCCSSCCOC(=O)Cc2ccc(OC(=O)CCCSSC)cc2)CCN2CCCC2)cc1] |
| com- pound 30 | 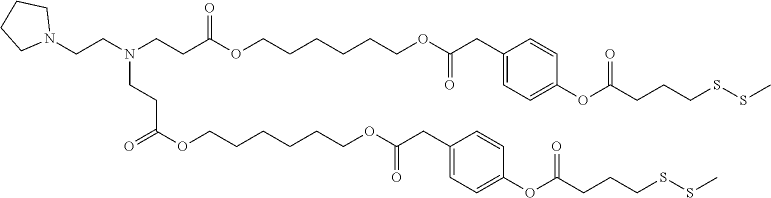 [SMILES: CSSCCCC(=O)Oc1ccc(CC(=O)OCCCCCCOC(=O)CCN(CCC(=O)OCCCCCCOC(=O)Cc2ccc(OC(=O)CCCSSC)cc2)CCN2CCCC2)cc1] |

TABLE 2

| name | structure |
|---|---|
| com- pari- son com- pound | 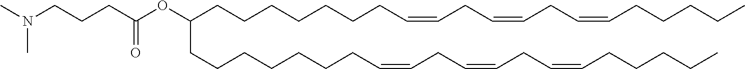 [SMILES: CCCCC/C=C\C/C=C\C/C=C\CCCCCCCC(CCCCCCC/C=C\C/C=C\C/C=C\CCCCC)OC(=O)CCCN(C)C] |

### [Example 1] Synthesis of Compound 1

Compound 1 was synthesized by the following synthesis route.

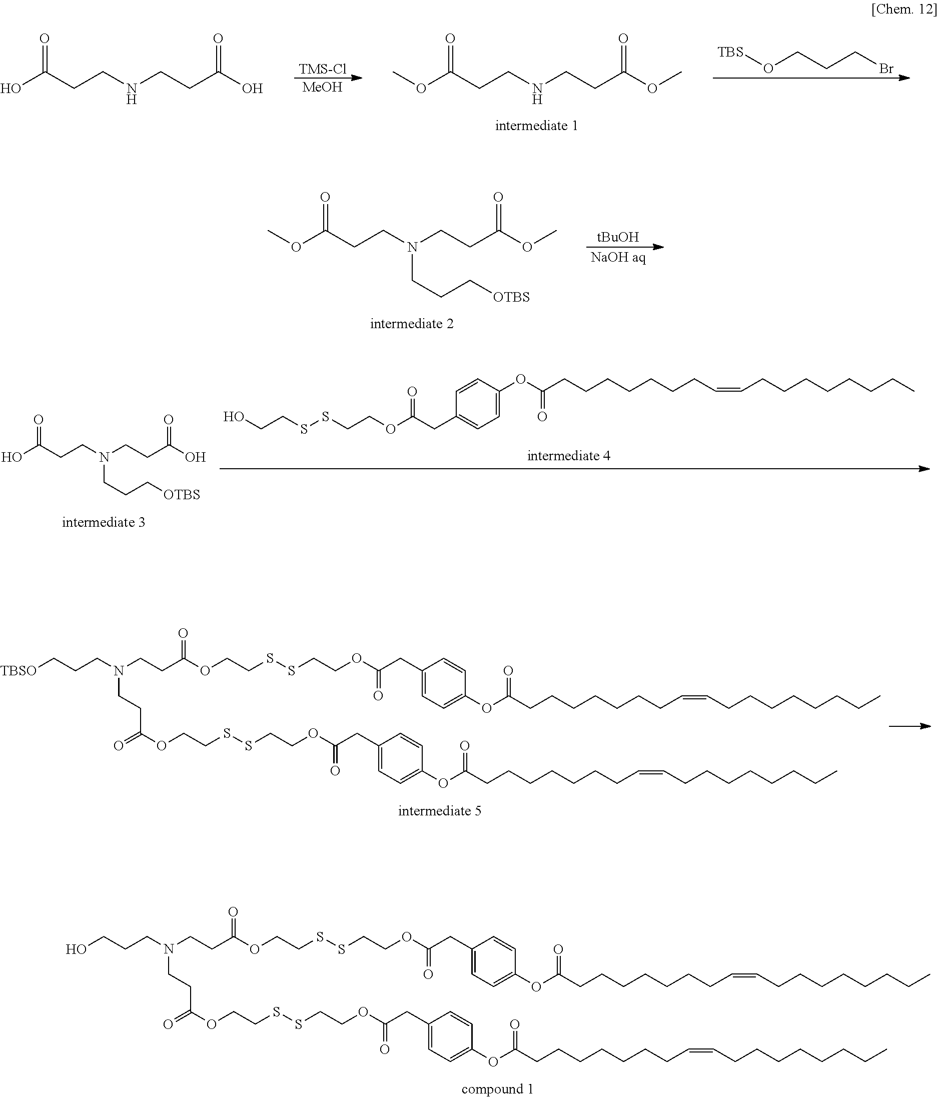

### <Synthesis of Intermediate 1>

3,3′-Iminodipropionic acid (500 mg, 3.10 mmol) was dissolved in methanol (3.50 g), TMS-Cl (674 mg, 6.20 mmol) was added, and the mixture was reacted at room temperature for 1 hr 30 min. The solution after the reaction was concentrated using an evaporator, and the concentrate was vacuum dried to give 666 mg of intermediate 1.

### <Synthesis of Intermediate 2>

Intermediate 1 (500 mg, 2.22 mmol) was dissolved in DMF (5.00 g), triethylamine (560 mg, 5.55 mmol), potassium iodide (36.8 mg, 0.022 mmol), and (3-bromopropoxy) (tert-butyl)dimethylsilane (561 mg, 2.22 mmol) were added, and the mixture was reacted at 70° C. for 11 hr. To the solution after the reaction was added chloroform (7.50 g), and the mixture was washed with 5 wt % sodium dihydrogen phosphate aqueous solution, 7 wt % sodium hydrogen carbonate aqueous solution, and 20 wt % sodium chloride aqueous solution, and dehydrated with sodium sulfate, and the filtrate after dehydration was concentrated using an evaporator. After concentration, silica gel column purification was performed using ethanol/chloroform to give 548 mg of intermediate 2.

### <Synthesis of Intermediate 3>

Intermediate 2 (200 mg, 0.528 mmol) was dissolved in tBuOH (1.38 g), 100 g/mL sodium hydroxide aqueous solution (845 mg) was added and the mixture was reacted at room temperature for 2 hr. The solution after the reaction was neutralized with 1.0 M sodium dihydrogen phosphate aqueous solution, acetonitrile (8.60 g) was added, and the mixture was concentrated using an evaporator. The concentrate was vacuum dried to give intermediate 3.

Intermediate 4 was synthesized by the following synthesis route.

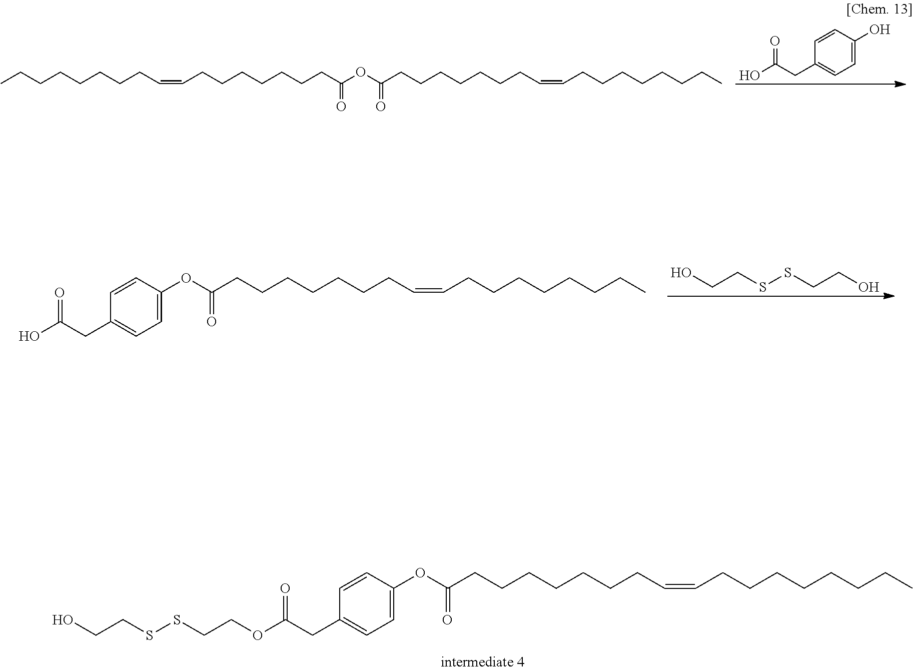

<Synthesis of 4-oleoyloxyphenylacetic acid>

Oleic anhydride (43.1 g, 78.9 mmol) and 4-hydroxyphenylacetic acid (6.00 g, 39.4 mmol) were dissolved in chloroform (647 g). Thereto was added DMAP (1.93 g, 15.8 mmol), and the mixture was reacted at room temperature for 9 hr. The reaction mixture was washed with 10 wt % acetic acid aqueous solution (216 g) and ion exchange water (216 g), magnesium sulfate (12.9 g) was added thereto, and the mixture was stirred for 30 min. The reaction mixture was dehydrated with magnesium sulfate, magnesium sulfate was removed by filtration, and the obtained filtrate was concentrated using an evaporator. The concentrate was dissolved in hexane (284 g), insoluble material was removed by filtration, and the obtained filtrate was extracted with acetonitrile (168 g). After extraction, the acetonitrile layer was concentrated using an evaporator. The obtained concentrate was subjected to silica gel column purification using ethanol/chloroform to give 3.80 g of 4-oleyloxyphenylacetic acid.

### <Synthesis of Intermediate 4>

4-Oleoyloxyphenylacetic acid (3.80 g, 9.12 mmol) was dissolved in chloroform (22.2 g), bis(2-hydroxyethyl)disulfide (1.48 g, 9.60 mmol), DMAP (235 mg, 1.92 mmol) and EDC hydrochloride (4.60 g, 24.0 mmol) were added and the mixture was reacted at room temperature for 2 hr. The solution after the reaction was washed with 5 wt % sodium dihydrogen phosphate aqueous solution, 7 wt % sodium hydrogen carbonate aqueous solution, and 20 wt % sodium chloride aqueous solution, and dehydrated with sodium sulfate. The filtrate after dehydration was concentrated using an evaporator. After concentration, silica gel column purification was performed using ethanol/chloroform to give 2.12 g of intermediate 4.

### <Synthesis of Intermediate 5>

Intermediate 4 was dissolved in chloroform (22.2 g), bis(2-hydroxyethyl)disulfide (1.48 g, 9.60 mmol), DMAP (235 mg, 1.92 mmol) and EDC hydrochloride (4.60 g, 24.0 mmol) were added and the mixture was reacted at room temperature for 2 hr. The solution after the reaction was washed with 5 wt % sodium dihydrogen phosphate aqueous solution, 7 wt % sodium hydrogen carbonate aqueous solution, and 20 wt % sodium chloride aqueous solution, and dehydrated with sodium sulfate. The filtrate after dehydration was concentrated using an evaporator. After concentration, silica gel column purification was performed using ethanol/chloroform to give 392 mg of intermediate 5.

### <Synthesis of Compound 1>

To intermediate 5 (285 mg, 0.203 mmol) was added isopropyl alcohol (4.56 g), p-toluenesulfonic acid monohydrate (158 mg, 0.831 mmol) was added, and the mixture was reacted at room temperature for 1 hr. To the solution after the reaction was added chloroform (4.56 g), and the mixture was washed with 7 wt % sodium hydrogen carbonate aqueous solution and 20 wt % sodium chloride aqueous solution. The mixture was dehydrated with sodium sulfate and the filtrate after dehydrating was concentrated using an evaporator. After concentration, silica gel column purification was performed using ethanol/chloroform to give 170 mg of compound 1.

<¹H-NMR (600 MHz, DMSO-d₆) of Compound 1>

δ: 0.79-0.87 (t, 6H), 1.17-1.38 (m, 42H), 1.44-1.55 (m, 2H), 1.57-1.66 (t, 4H), 1.92-2.04 (t, 8H), 2.34-2.44 (m, 6H), 2.51-2.59 (m, 5H), 2.63-2.68 (m, 2H), 2.91-3.02 (m, 8H), 3.34-3.40 (m, 2H), 3.69 (s, 4H), 4.18-4.32 (m, 8H), 5.29-5.37 (m, 4H), 7.00-7.06 (m, 4H), 7.27-7.34 (m, 4H)

### [Example 2] Synthesis of Compound 2

Compound 2 was synthesized by the following synthesis route.

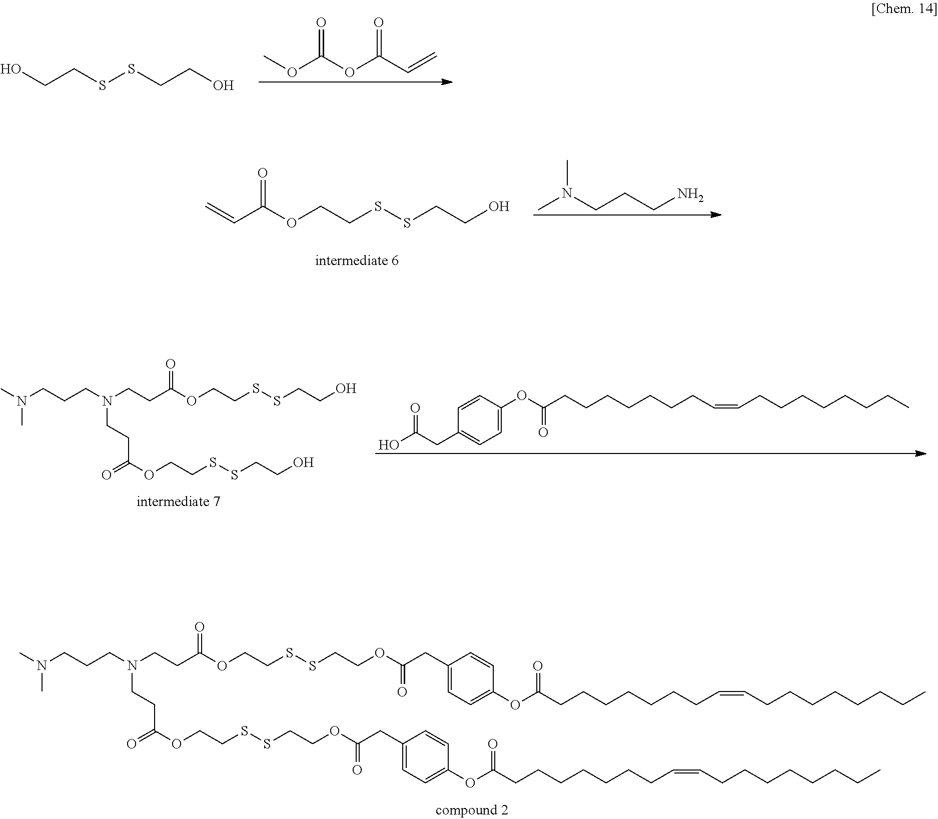

### <Synthesis of Intermediate 6>

Bis(2-hydroxyethyl)disulfide (1.15 g, 7.55 mmol) was dissolved in chloroform (11.5 g), acrylic anhydride (893 mg, 7.08 mmol) and triethylamine (2.26 g, 22.3 mmol) were added, and the mixture was reacted at room temperature for 1 hr. The solution after the reaction was washed with 5 wt % sodium dihydrogen phosphate aqueous solution, 7 wt % sodium hydrogen carbonate aqueous solution, and 20 wt % sodium chloride aqueous solution, and dehydrated with sodium sulfate. The filtrate after dehydration was concentrated using an evaporator. After concentration, silica gel column purification was performed using ethanol/chloroform to give 670 mg of intermediate 6.

### <Synthesis of Intermediate 7>

To N,N-dimethyl-1,3-propanediamine (30.0 mg, 0.294 mmol) was added intermediate 6 (153 mg, 0.735 mmol) and the mixture was reacted at room temperature for 10 hr to give 193 mg of intermediate 7 as a crude product.

### <Synthesis of Compound 2>

The crude product (193 mg, 0.372 mmol) of intermediate 7 was dissolved in chloroform (1.93 g), 4-oleoyloxyphenylacetic acid (387 mg, 0.929 mmol), DMAP (9.1 mg, 0.074 mmol), and EDC hydrochloride (214 mg, 1.12 mmol) were added, and the mixture was reacted at room temperature for 2 hr. The solution after the reaction was subjected to silica gel column purification using ethanol/chloroform to give 230 mg of compound 2.

<¹H-NMR (600 MHz, DMSO-d₆) of Compound 2>

δ: 0.81-0.88 (t, 6H), 1.17-1.38 (m, 42H), 1.40-1.48 (m, 2H), 1.59-1.66 (t, 4H), 1.92-2.04 (t, 8H), 2.05-2.15 (m, 6H), 2.31-2.42 (m, 6H), 2.51-2.59 (m, 4H), 2.61-2.68 (m, 4H), 2.90-3.02 (m, 8H), 3.69 (s, 4H), 4.18-4.32 (m, 8H), 5.29-5.37 (m, 4H), 7.00-7.06 (m, 4H), 7.27-7.34 (m, 4H)

### [Example 3] Synthesis of Compound 3

### <Synthesis of Intermediate 8>

Using intermediate 6 (142 mg, 0.675 mmol) and N,N-diethyl-1,3-diaminopropane (40.0 mg, 0.307 mmol) and in the same manner as in Synthesis of intermediate 7 described in Example 2, 182 mg of intermediate 8 was synthesized.

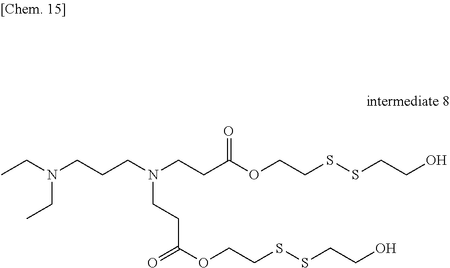 [SMILES: CCN(CC)CCCN(CCC(=O)OCCSSCCO)CCC(=O)OCCSSCCO]

### <Synthesis of Compound 3>

Using 4-oleoyloxyphenylacetic acid (381 mg, 0.915 mmol) and intermediate 8 (182 mg, 0.333 mmol) and in the same manner as in Synthesis of compound 2 described in Example 2, 150 mg of compound 3 was synthesized.

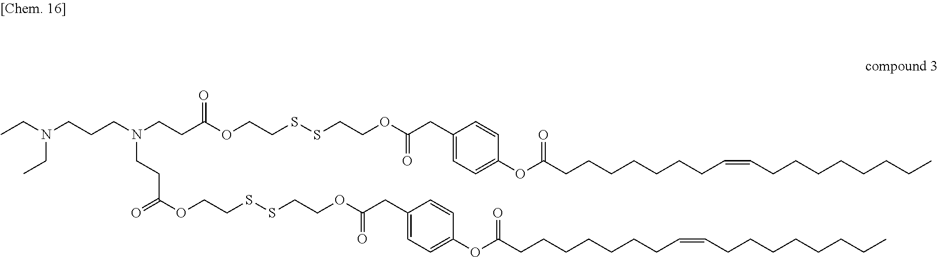 [SMILES: CCCCCCCC/C=C\CCCCCCCC(=O)Oc1ccc(CC(=O)OCCSSCCOC(=O)CCN(CCCN(CC)CC)CCC(=O)OCCSSCCOC(=O)Cc2ccc(OC(=O)CCCCCCC/C=C\CCCCCCCC)cc2)cc1]

<¹H-NMR (600 MHz, DMSO-d₆) of Compound 3>

δ: 0.81-0.88 (t, 6H), 0.89-0.95 (m, 6H), 1.17-1.38 (m, 42H), 1.40-1.48 (m, 2H), 1.59-1.66 (m, 4H), 1.92-2.04 (t, 8H), 2.28-2.30 (m, 2H), 2.31-2.42 (m, 8H), 2.51-2.59 (m, 4H), 2.61-2.68 (m, 4H), 2.90-3.02 (m, 8H), 3.69 (s, 4H), 4.18-4.32 (m, 8H), 5.29-5.37 (m, 4H), 7.00-7.06 (m, 4H), 7.27-7.34 (m, 4H)

### [Example 4] Synthesis of Compound 4

### <Synthesis of Intermediate 9>

Using intermediate 6 (156 mg, 0.748 mmol) and N,N-dimethylethylenediamine (30.0 mg, 0.340 mmol) and in the same manner as in Synthesis of intermediate 7 described in Example 2, 186 mg of intermediate 9 was synthesized.

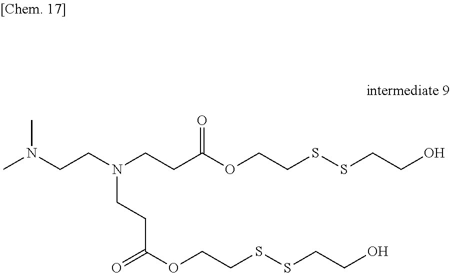 [SMILES: CN(C)CCN(CCC(=O)OCCSSCCO)CCC(=O)OCCSSCCO]

### <Synthesis of Compound 4>

Using 4-oleoyloxyphenylacetic acid (381 mg, 0.915 mmol) and intermediate 9 (186 mg, 0.369 mmol) and in the same manner as in Synthesis of compound 2 described in Example 2, 140 mg of compound 4 was synthesized.

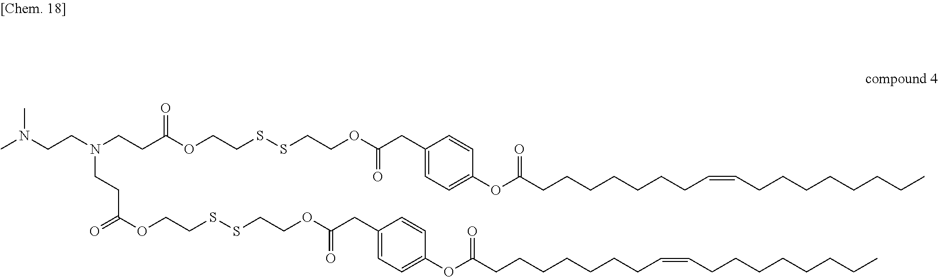 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN(C)C)cc1]

<¹H-NMR (600 MHz, DMSO-d₆) of Compound 4>

δ: 0.81-0.88 (t, 6H), 1.17-1.38 (m, 42H), 1.59-1.66 (t, 4H), 1.92-2.04 (t, 8H), 2.05-2.15 (m, 4H), 2.18-2.24 (m, 2H), 2.38-2.42 (m, 6H), 2.51-2.59 (m, 4H), 2.61-2.68 (m, 4H), 2.90-3.02 (m, 8H), 3.69 (s, 4H), 4.18-4.32 (m, 8H), 5.29-5.37 (m, 4H), 7.00-7.06 (m, 4H), 7.27-7.34 (m, 4H)

### [Example 5] Synthesis of Compound 5

### <Synthesis of Intermediate 10>

Using intermediate 6 (161 mg, 0.773 mmol) and 1-(3-aminopropyl)pyrrolidine (45.0 mg, 0.351 mmol) and in the same manner as in Synthesis of intermediate 7 described in Example 2, 206 mg of intermediate 10 was synthesized.

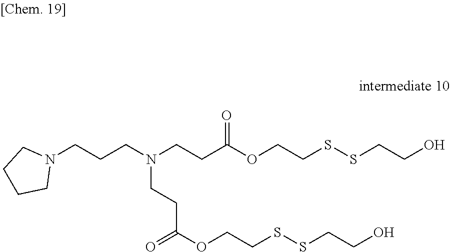 [SMILES: O=C(CCN(CCCN1CCCC1)CCC(=O)OCCSSCCO)OCCSSCCO]

### <Synthesis of Compound 5>

Using 4-oleoyloxyphenylacetic acid (354 mg, 0.850 mmol) and intermediate 10 (206 mg, 0.378 mmol) and in the same manner as in Synthesis of compound 2 described in Example 2, 157 mg of compound 5 was synthesized.

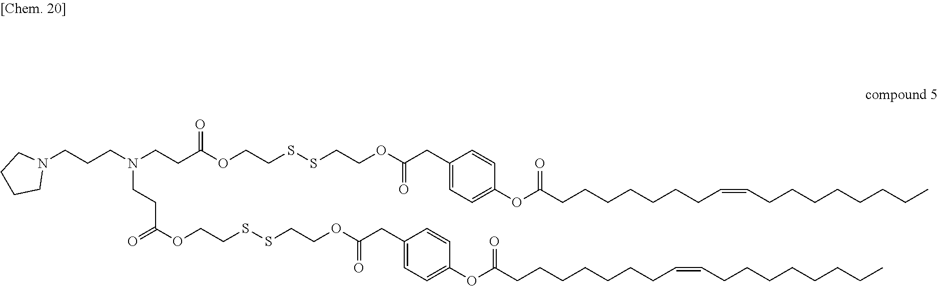 [SMILES: CCCCCCCC/C=C\CCCCCCCC(=O)Oc1ccc(CC(=O)OCCSSCCOC(=O)CCN(CCCN2CCCC2)CCC(=O)OCCSSCCOC(=O)Cc2ccc(OC(=O)CCCCCCC/C=C\CCCCCCCC)cc2)cc1]

<¹H-NMR (400 MHz, DMSO-d₆) of Compound 5>

δ: 0.81-0.88 (t, 6H), 1.17-1.38 (m, 42H), 1.42-1.52 (m, 2H), 1.59-1.66 (m, 8H), 1.92-2.04 (m, 8H), 2.26-2.44 (m, 12H), 2.51-2.59 (m, 4H), 2.61-2.68 (m, 4H), 2.90-3.02 (m, 8H), 3.69 (s, 4H), 4.18-4.32 (m, 8H), 5.29-5.37 (m, 4H), 7.00-7.06 (m, 4H), 7.27-7.34 (m, 4H)

### [Example 6] Synthesis of Compound 6

### <Synthesis of Intermediate 11>

Using intermediate 6 (150 mg, 0.720 mmol) and tryptamine (52.5 mg, 0.328 mmol) and in the same manner as in Synthesis of intermediate 7 described in Example 2, 203 mg of intermediate 11 was synthesized.

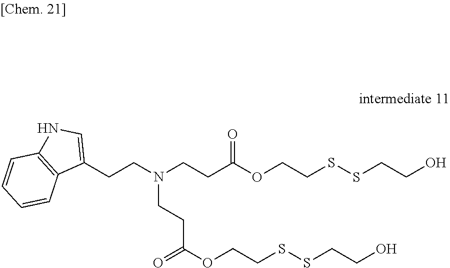 [SMILES: O=C(CCN(CCC(=O)OCCSSCCO)CCc1c[nH]c2ccccc12)OCCSSCCO]

### <Synthesis of Compound 6>

Using 4-oleoyloxyphenylacetic acid (330 mg, 0.792 mmol) and intermediate 11 (203 mg, 0.351 mmol) and in the same manner as in Synthesis of compound 2 described in Example 2, 231 mg of compound 6 was synthesized.

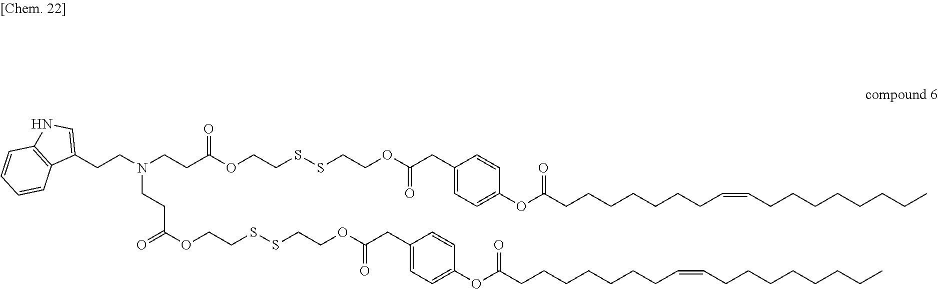 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCc2c[nH]c3ccccc23)cc1]

<¹H-NMR (400 MHz, DMSO-d₆) of Compound 6>

δ: 0.81-0.88 (t, 6H), 1.17-1.38 (m, 42H), 1.57-1.66 (m, 4H), 1.92-2.04 (m, 8H), 2.40-2.48 (m, 4H), 2.51-2.59 (m, 2H), 2.61-2.83 (m, 8H), 2.91-3.01 (m, 8H), 3.69 (s, 4H), 4.18-4.32 (m, 8H), 5.29-5.37 (m, 4H), 6.90-6.98 (m, 1H), 7.00-7.06 (m, 5H), 7.08-7.13 (m, 1H), 7.27-7.34 (m, 5H), 7.48-7.52 (m, 1H)

### [Example 7] Synthesis of Compound 7

### <Synthesis of Intermediate 12>

Using intermediate 6 (155 mg, 0.744 mmol) and 4-(2-aminoethyl)morpholine (44.0 mg, 0.338 mmol) and in the same manner as in Synthesis of intermediate 7 described in Example 2, 199 mg of intermediate 12 was synthesized.

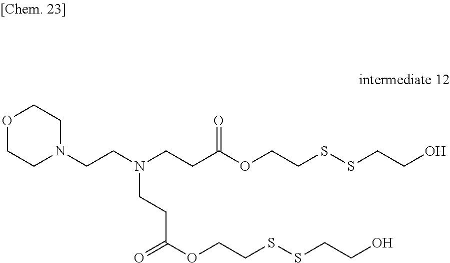 [SMILES: O=C(CCN(CCC(=O)OCCSSCCO)CCN1CCOCC1)OCCSSCCO]

### <Synthesis of Compound 7>

Using 4-oleoyloxyphenylacetic acid (414 mg, 0.994 mmol) and intermediate 12 (199 mg, 0.364 mmol) and in the same manner as in Synthesis of compound 2 described in Example 2, 140 mg of compound 7 was synthesized.

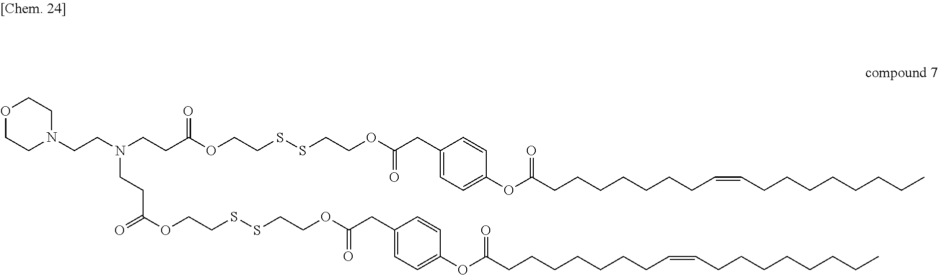 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN2CCOCC2)cc1]

<¹H-NMR (400 MHz, DMSO-d₆) of Compound 7>

δ: 0.81-0.88 (t, 6H), 1.17-1.38 (m, 42H), 1.59-1.66 (m, 4H), 1.92-2.04 (m, 8H), 2.26-2.44 (m, 8H), 2.54-2.59 (m, 6H), 2.63-2.73 (m, 4H), 2.90-3.02 (m, 8H), 3.48-3.56 (m, 4H), 3.69 (s, 4H), 4.18-4.32 (m, 8H), 5.29-5.37 (m, 4H), 7.00-7.06 (m, 4H), 7.27-7.34 (m, 4H)

### [Example 8] Synthesis of Compound 8

### <Synthesis of Intermediate 13>

Using intermediate 6 (156 mg, 0.749 mmol) and 1-(3-aminopropyl)imidazole (42.5 mg, 0.340 mmol) and in the same manner as in Synthesis of intermediate 7 described in Example 2, 199 mg of intermediate 13 was synthesized.

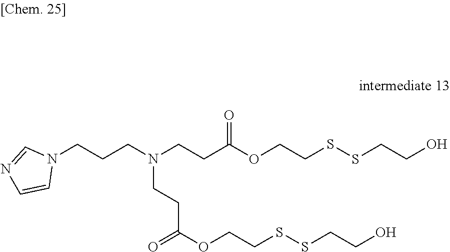 [SMILES: O=C(CCN(CCCn1ccnc1)CCC(=O)OCCSSCCO)OCCSSCCO]

### <Synthesis of Compound 8>

Using 4-oleoyloxyphenylacetic acid (502 mg, 1.20 mol) and intermediate 13 (199 mg, 0.367 mmol) and in the same manner as in Synthesis of compound 2 described in Example 2, 39 mg of compound 8 was synthesized.

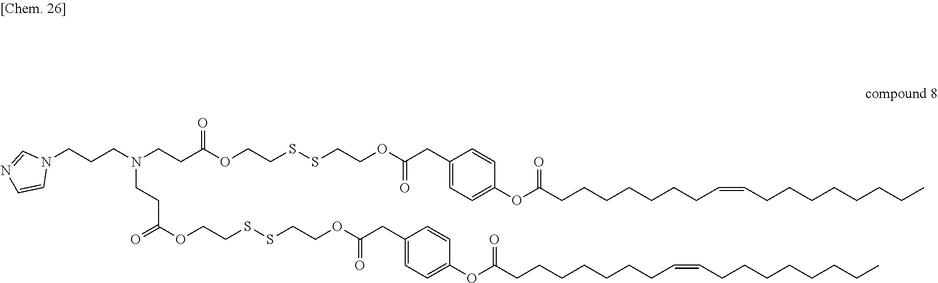 [SMILES: CCCCCCCC/C=C\CCCCCCCC(=O)Oc1ccc(CC(=O)OCCSSCCOC(=O)CCN(CCCn2ccnc2)CCC(=O)OCCSSCCOC(=O)Cc2ccc(OC(=O)CCCCCCC/C=C\CCCCCCCC)cc2)cc1]

<¹H-NMR (400 MHz, DMSO-d₆) of Compound 8>

δ: 0.81-0.88 (t, 6H), 1.17-1.38 (m, 42H), 1.59-1.66 (m, 4H), 1.83-1.95 (m, 2H), 1.92-2.04 (m, 8H), 2.33-2.46 (m, 6H), 2.51-2.59 (m, 4H), 2.63-2.73 (m, 4H), 2.90-3.02 (m, 8H), 3.69 (s, 4H), 4.18-4.32 (m, 8H), 5.29-5.37 (m, 4H), 6.95 (s, 1H), 7.00-7.06 (m, 5H), 7.27-7.34 (m, 4H), 7.51 (s, 1H)

### [Example 9] Synthesis of Compound 9

### <Synthesis of Intermediate 14>

Using intermediate 6 (156 mg, 0.748 mmol) and N,N-diethylethylenediamine (39.5 mg, 0.340 mmol) and in the same manner as in Synthesis of intermediate 7 described in Example 2, 196 mg of intermediate 14 was synthesized.

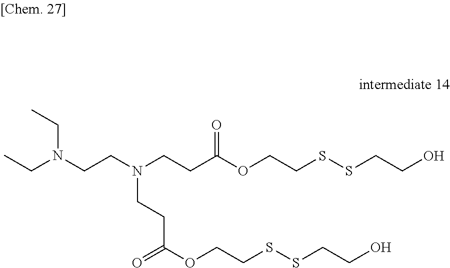 [SMILES: CCN(CC)CCN(CCC(=O)OCCSSCCO)CCC(=O)OCCSSCCO]

### <Synthesis of Compound 9>

Using 4-oleoyloxyphenylacetic acid (343 mg, 0.823 mmol) and intermediate 14 (196 mg, 0.347 mmol) and in the same manner as in Synthesis of compound 2 described in Example 2, 150 mg of compound 9 was synthesized.

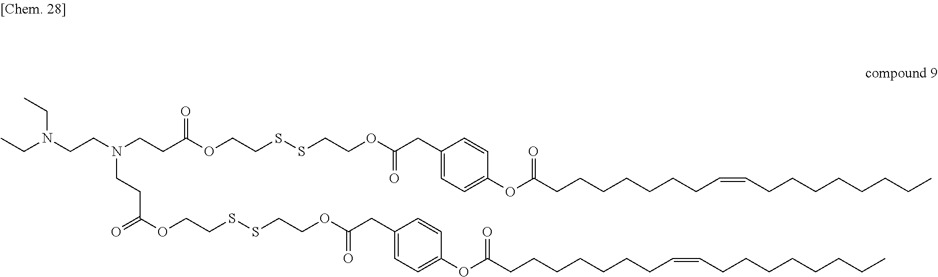 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN(CC)CC)cc1]

<¹H-NMR (600 MHz, DMSO-d₆) of Compound 9>

δ: 0.81-0.88 (t, 6H), 0.89-0.95 (m, 6H), 1.17-1.38 (m, 40H), 1.59-1.66 (m, 4H), 2.14-2.18 (t, 8H), 2.51-2.59 (m, 16H), 2.90-3.02 (m, 8H), 3.69 (s, 4H), 3.70-3.74 (m, 4H), 4.18-4.32 (m, 8H), 5.29-5.37 (m, 4H), 7.00-7.06 (m, 4H), 7.27-7.34 (m, 4H)

### [Example 10] Synthesis of Compound 10

### <Synthesis of Intermediate 15>

Using intermediate 6 (156 mg, 0.748 mmol) and 3-(di-n-propylamino)propylamine (49.0 mg, 0.340 mmol) and in the same manner as in Synthesis of intermediate 7 described in Example 2, 205 mg of intermediate 15 was synthesized.

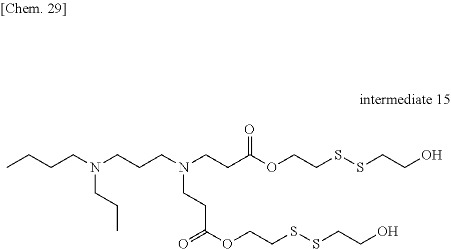 [SMILES: CCCCN(CCC)CCCN(CCC(=O)OCCSSCCO)CCC(=O)OCCSSCCO]

### <Synthesis of Compound 10>

Using 4-oleoyloxyphenylacetic acid (343 mg, 0.823 mmol) and intermediate 15 (205 mg, 0.366 mmol) and in the same manner as in Synthesis of compound 2 described in Example 2, 147 mg of compound 10 was synthesized.

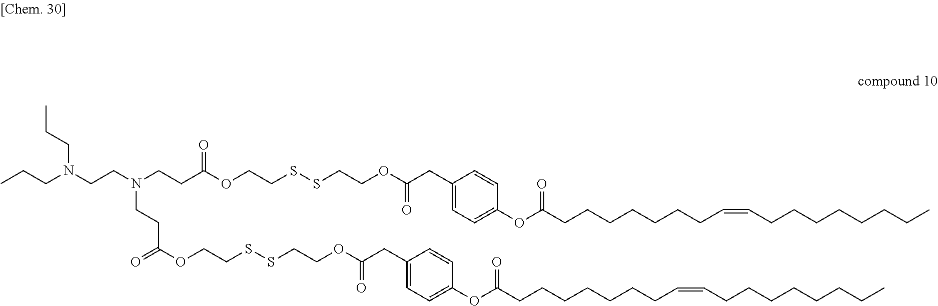 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN(CCC)CCC)cc1]

<¹H-NMR (600 MHz, DMSO-d₆) of Compound 10>

δ: 0.81-0.89 (m, 12H), 1.17-1.38 (m, 40H), 1.43-1.45 (m, 4H), 1.59-1.66 (m, 4H), 2.14-2.18 (t, 8H), 2.51-2.59 (m, 16H), 2.90-3.02 (m, 8H), 3.69 (s, 4H), 3.70-3.74 (m, 4H), 4.18-4.32 (m, 8H), 5.29-5.37 (m, 4H), 7.00-7.06 (m, 4H), 7.27-7.34 (m, 4H)

### [Example 11] Synthesis of Compound 11

### <Synthesis of Intermediate 16>

Using intermediate 6 (156 mg, 0.748 mmol) and 3-(di-n-propylamino)propylamine (49.0 mg, 0.340 mmol) and in the same manner as in Synthesis of intermediate 7 described in Example 2, 205 mg of intermediate 16 was synthesized.

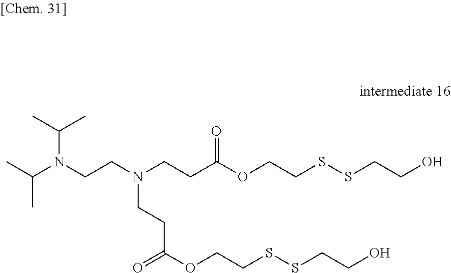 [SMILES: CC(C)N(CCN(CCC(=O)OCCSSCCO)CCC(=O)OCCSSCCO)C(C)C]

### <Synthesis of Compound 11>

Using 4-oleoyloxyphenylacetic acid (343 mg, 0.823 mmol) and intermediate 16 (205 mg, 0.366 mmol) and in the same manner as in Synthesis of compound 2 described in Example 2, 149 mg of compound 11 was synthesized.

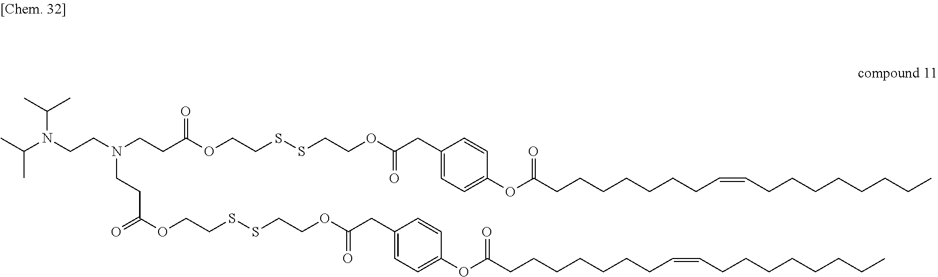 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN(C(C)C)C(C)C)cc1]

<¹H-NMR (600 MHz, DMSO-d₆) of Compound 11>

δ: 0.81-0.89 (m, 6H), 0.98-1.01 (m, 12H), 1.17-1.38 (m, 40H), 1.59-1.66 (m, 4H), 2.14-2.18 (t, 8H), 2.51-2.59 (m, 14H), 2.90-3.02 (m, 8H), 3.69 (s, 4H), 3.70-3.74 (m, 4H), 4.18-4.32 (m, 8H), 5.29-5.37 (m, 4H), 7.00-7.06 (m, 4H), 7.27-7.34 (m, 4H)

### [Example 12] Synthesis of Compound 12

### <Synthesis of Intermediate 17>

Using intermediate 6 (156 mg, 0.748 mmol) and (2-aminoethyl) (ethyl)methylamine (34.7 mg, 0.340 mmol) and in the same manner as in Synthesis of intermediate 7 described in Example 2, 191 mg of intermediate 17 was synthesized.

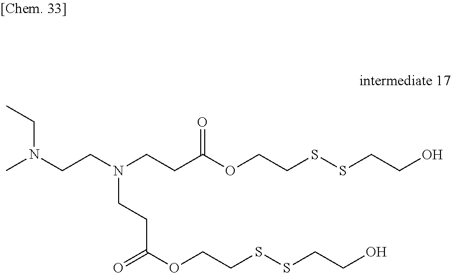 [SMILES: CCN(C)CCN(CCC(=O)OCCSSCCO)CCC(=O)OCCSSCCO]

### <Synthesis of Compound 12>

Using 4-oleoyloxyphenylacetic acid (343 mg, 0.823 mmol) and intermediate 17 (191 mg, 0.368 mmol) and in the same manner as in Synthesis of compound 2 described in Example 2, 142 mg of compound 12 was synthesized.

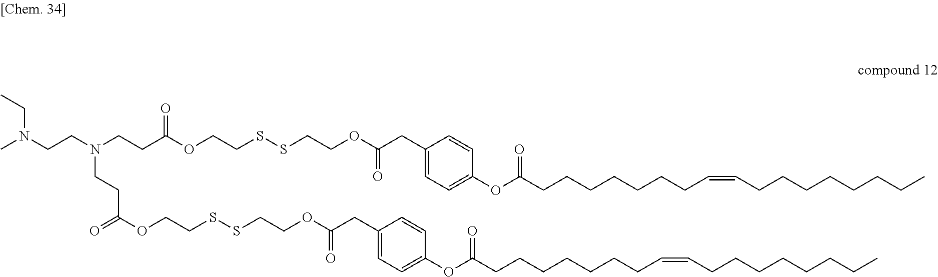 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN(C)CC)cc1]

<¹H-NMR (600 MHz, DMSO-d₆) of Compound 12>

δ: 0.81-0.89 (m, 6H), 0.98-1.01 (m, 3H), 1.17-1.38 (m, 40H), 1.59-1.66 (m, 4H), 2.14-2.18 (t, 11H), 2.51-2.59 (m, 14H), 2.90-3.02 (m, 8H), 3.69 (s, 4H), 3.70-3.74 (m, 4H), 4.18-4.32 (m, 8H), 5.29-5.37 (m, 4H), 7.00-7.06 (m, 4H), 7.27-7.34 (m, 4H)

### [Example 13] Synthesis of Compound 13

### <Synthesis of Intermediate 18>

Using intermediate 6 (156 mg, 0.748 mmol) and (2-aminoethyl) (methyl)propylamine (39.5 mg, 0.340 mmol) and in the same manner as in Synthesis of intermediate 7 described in Example 2, 196 mg of intermediate 18 was synthesized.

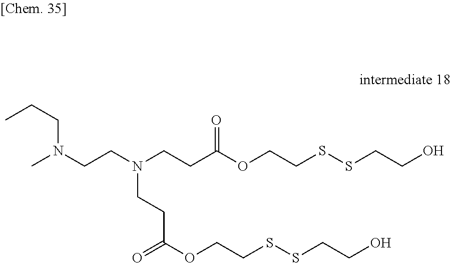 [SMILES: CCCN(C)CCN(CCC(=O)OCCSSCCO)CCC(=O)OCCSSCCO]

### <Synthesis of Compound 13>

Using 4-oleoyloxyphenylacetic acid (343 mg, 0.823 mmol) and intermediate 18 (196 mg, 0.368 mmol) and in the same manner as in Synthesis of compound 2 described in Example 2, 146 mg of compound 13 was synthesized.

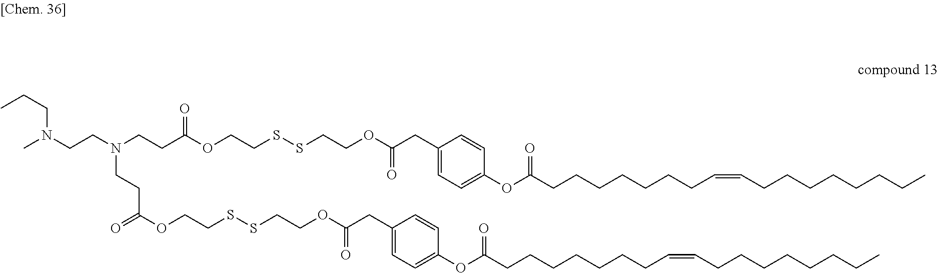 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN(C)CCC)cc1]

<¹H-NMR (600 MHz, DMSO-d₆) of Compound 13>

δ: 0.81-0.89 (m, 9H), 1.17-1.38 (m, 42H), 1.59-1.66 (m, 4H), 2.14-2.18 (t, 11H), 2.51-2.59 (m, 14H), 2.90-3.02 (m, 8H), 3.69 (s, 4H), 3.70-3.74 (m, 4H), 4.18-4.32 (m, 8H), 5.29-5.37 (m, 4H), 7.00-7.06 (m, 4H), 7.27-7.34 (m, 4H)

### [Example 14] Synthesis of Compound 14

### <Synthesis of Intermediate 19>

Using intermediate 6 (156 mg, 0.748 mmol) and 2-(2-aminoethyl)-1-methylpyrrolidine (43.6 mg, 0.340 mmol) and in the same manner as in Synthesis of intermediate 7 described in Example 2, 200 mg of intermediate 19 was synthesized.

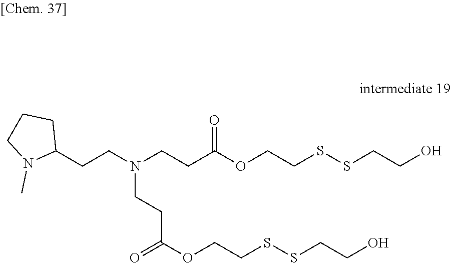 [SMILES: CN1CCCC1CCN(CCC(=O)OCCSSCCO)CCC(=O)OCCSSCCO]

### <Synthesis of Compound 14>

Using 4-oleoyloxyphenylacetic acid (343 mg, 0.823 mmol) and intermediate 19 (200 mg, 0.367 mmol) and in the same manner as in Synthesis of compound 2 described in Example 2, 150 mg of compound 14 was synthesized.

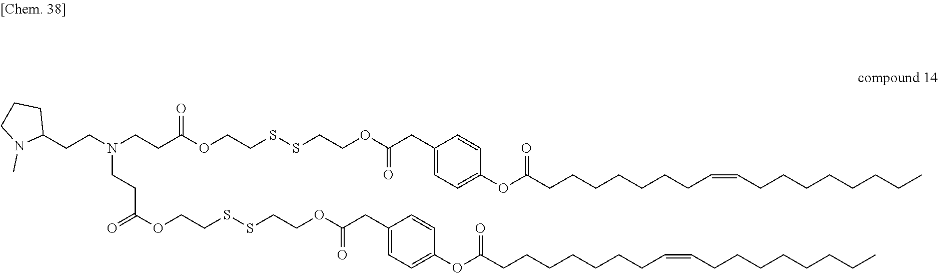 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCC2CCCN2C)cc1]

<¹H-NMR (600 MHz, DMSO-d₆) of Compound 14>

δ: 0.81-0.89 (m, 6H), 1.17-1.38 (m, 40H), 1.40-1.47 (m, 4H), 1.59-1.66 (m, 6H), 2.14-2.18 (t, 8H), 2.25-2.30 (m, 5H), 2.40-2.46 (m, 3H), 2.51-2.59 (m, 8H), 2.90-3.02 (m, 8H), 3.69 (s, 4H), 3.70-3.74 (m, 4H), 4.18-4.32 (m, 8H), 5.29-5.37 (m, 4H), 7.00-7.06 (m, 4H), 7.27-7.34 (m, 4H)

### [Example 15] Synthesis of Compound 15

### <Synthesis of Intermediate 20>

Using intermediate 6 (156 mg, 0.748 mmol) and 2-(4-methylpiperazin-1-yl)ethane-1-amine (48.7 mg, 0.340 mmol) and in the same manner as in Synthesis of intermediate 7 described in Example 2, 205 mg of intermediate 20 was synthesized.

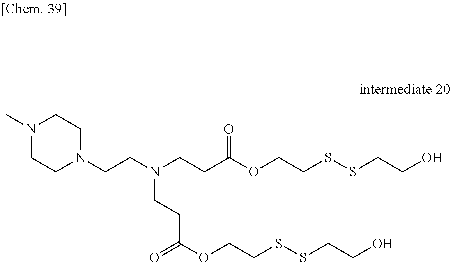 [SMILES: CN1CCN(CCN(CCC(=O)OCCSSCCO)CCC(=O)OCCSSCCO)CC1]

### <Synthesis of Compound 15>

Using 4-oleoyloxyphenylacetic acid (343 mg, 0.823 mmol) and intermediate 20 (205 mg, 0.366 mmol) and in the same manner as in Synthesis of compound 2 described in Example 2, 152 mg of compound 15 was synthesized.

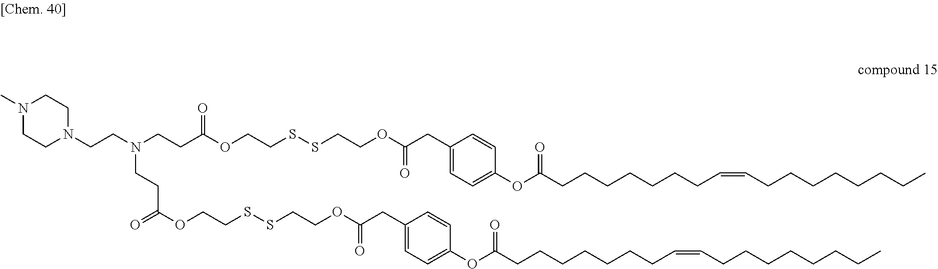 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN2CCN(C)CC2)cc1]

<¹H-NMR (600 MHz, DMSO-d₆) of Compound 15>

δ: 0.81-0.89 (m, 6H), 1.17-1.38 (m, 40H), 1.59-1.66 (m, 4H), 2.14-2.18 (m, 11H), 2.25-2.30 (m, 8H), 2.40-2.46 (m, 4H), 2.51-2.59 (m, 8H), 2.90-3.02 (m, 8H), 3.69 (s, 4H), 3.70-3.74 (m, 4H), 4.18-4.32 (m, 8H), 5.29-5.37 (m, 4H), 7.00-7.06 (m, 4H), 7.27-7.34 (m, 4H)

### [Example 16] Synthesis of Compound 16

### <Synthesis of Intermediate 21>

Using intermediate 6 (156 mg, 0.748 mmol) and 4-methyl-1-piperazinepropane-1-amine (53.5 mg, 0.340 mmol) and in the same manner as in Synthesis of intermediate 7 described in Example 2, 210 mg of intermediate 21 was synthesized.

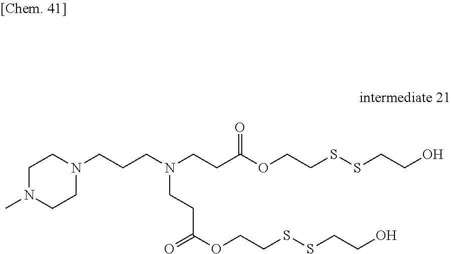 [SMILES: CN1CCN(CCCN(CCC(=O)OCCSSCCO)CCC(=O)OCCSSCCO)CC1]

### <Synthesis of Compound 16>

Using 4-oleoyloxyphenylacetic acid (343 mg, 0.823 mmol) and intermediate 21 (210 mg, 0.366 mmol) and in the same manner as in Synthesis of compound 2 described in Example 2, 155 mg of compound 16 was synthesized.

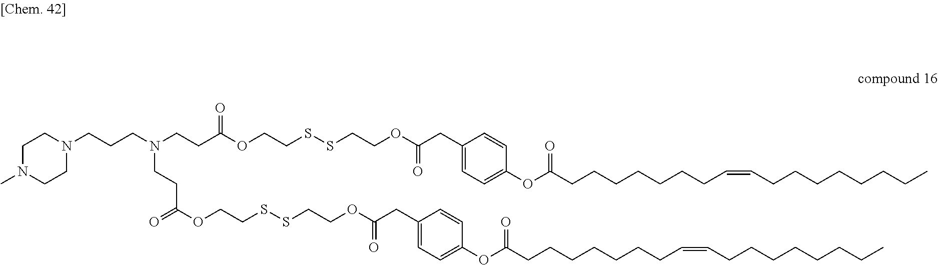 [SMILES: CCCCCCCC/C=C\CCCCCCCC(=O)Oc1ccc(CC(=O)OCCSSCCOC(=O)CCN(CCCN2CCN(C)CC2)CCC(=O)OCCSSCCOC(=O)Cc2ccc(OC(=O)CCCCCCC/C=C\CCCCCCCC)cc2)cc1]

<¹H-NMR (600 MHz, DMSO-d₆) of Compound 16>

δ: 0.81-0.89 (m, 6H), 1.17-1.68 (m, 46H), 2.14-2.18 (m, 11H), 2.25-2.30 (m, 8H), 2.40-2.46 (m, 4H), 2.51-2.59 (m, 8H), 2.90-3.02 (m, 8H), 3.69 (s, 4H), 3.70-3.74 (m, 4H), 4.18-4.32 (m, 8H), 5.29-5.37 (m, 4H), 7.00-7.06 (m, 4H), 7.27-7.34 (m, 4H)

### [Example 17] Synthesis of Compound 17

### <Synthesis of Intermediate 22>

Using intermediate 6 (156 mg, 0.748 mmol) and 2-(1H-pyrrol-1-yl)ethylamine (37.5 mg, 0.340 mmol) and in the same manner as in Synthesis of intermediate 7 described in Example 2, 194 mg of intermediate 22 was synthesized.

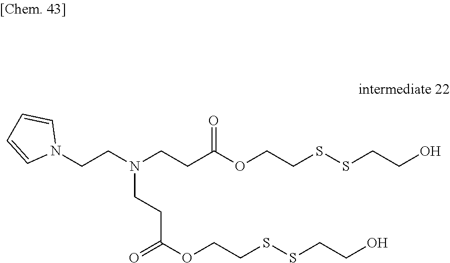 [SMILES: O=C(CCN(CCC(=O)OCCSSCCO)CCn1cccc1)OCCSSCCO]

### <Synthesis of Compound 17>

Using 4-oleoyloxyphenylacetic acid (343 mg, 0.823 mmol) and intermediate 22 (194 mg, 0.363 mmol) and in the same manner as in Synthesis of compound 2 described in Example 2, 149 mg of compound 17 was synthesized.

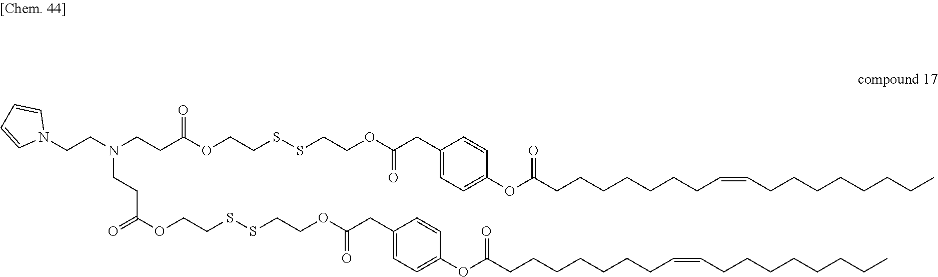 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCn2cccc2)cc1]

<¹H-NMR (600 MHz, DMSO-d₆) of Compound 17>

δ: 0.81-0.89 (m, 6H), 1.17-1.38 (m, 40H), 1.64-1.68 (m, 4H), 2.14-2.18 (m, 8H), 2.50-2.54 (m, 8H), 2.59-2.62 (m, 2H), 2.90-3.02 (m, 8H), 3.69 (s, 4H), 3.70-3.74 (m, 4H), 4.18-4.32 (m, 8H), 4.50-4.55 (m, 2H), 5.29-5.37 (m, 4H), 6.12-6.16 (m, 2H), 7.00-7.06 (m, 4H), 7.27-7.34 (m, 6H)

### [Example 18] Synthesis of Compound 18

### <Synthesis of Intermediate 23>

Using intermediate 6 (156 mg, 0.748 mmol) and 2-(1-azepanyl)ethaneamine (48.4 mg, 0.340 mmol) and in the same manner as in Synthesis of intermediate 7 described in Example 2, 204 mg of intermediate 23 was synthesized.

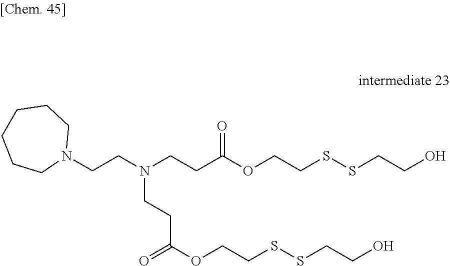 [SMILES: O=C(CCN(CCC(=O)OCCSSCCO)CCN1CCCCCC1)OCCSSCCO]

### <Synthesis of Compound 18>

Using 4-oleoyloxyphenylacetic acid (343 mg, 0.823 mmol) and intermediate 23 (204 mg, 0.366 mmol) and in the same manner as in Synthesis of compound 2 described in Example 2, 148 mg of compound 18 was synthesized.

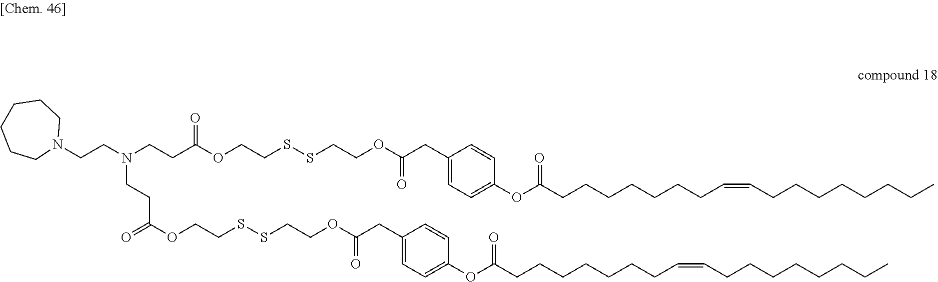 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN2CCCCCC2)cc1]

<¹H-NMR (600 MHz, DMSO-d₆) of Compound 18>

δ: 0.81-0.89 (m, 6H), 1.17-1.38 (m, 40H), 1.59-1.66 (m, 8H), 1.75-1.80 (m, 4H), 2.14-2.18 (m, 8H), 2.25-2.50 (m, 12H), 2.90-3.02 (m, 8H), 3.07-3.11 (m, 4H), 3.69 (s, 4H), 3.70-3.74 (m, 4H), 4.18-4.32 (m, 8H), 5.29-5.37 (m, 4H), 7.00-7.06 (m, 4H), 7.27-7.34 (m, 4H)

### [Example 19] Synthesis of Compound 19

### <Synthesis of Intermediate 24>

Using intermediate 6 (156 mg, 0.748 mmol) and 4-(2-aminoethyl)pyridine (41.5 mg, 0.340 mmol) and in the same manner as in Synthesis of intermediate 7 described in Example 2, 198 mg of intermediate 24 was synthesized.

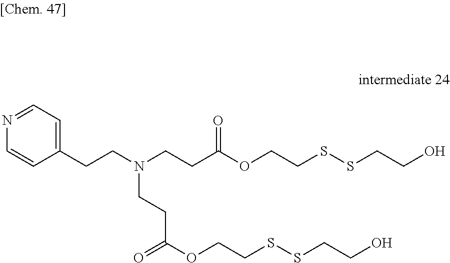 [SMILES: O=C(CCN(CCC(=O)OCCSSCCO)CCc1ccncc1)OCCSSCCO]

### <Synthesis of Compound 19>

Using 4-oleoyloxyphenylacetic acid (343 mg, 0.823 mmol) and intermediate 24 (198 mg, 0.368 mmol) and in the same manner as in Synthesis of compound 2 described in Example 2, 147 mg of compound 19 was synthesized.

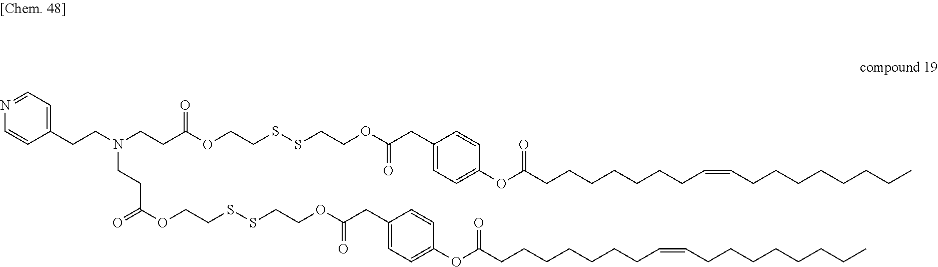 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCc2ccncc2)cc1]

<¹H-NMR (600 MHz, DMSO-d₆) of Compound 19>

δ: 0.81-0.89 (m, 6H), 1.17-1.38 (m, 40H), 1.59-1.66 (m, 4H), 2.14-2.18 (m, 8H), 2.48-2.54 (m, 8H), 2.68-2.73 (m, 2H), 2.90-3.02 (m, 8H), 3.44-3.46 (m, 2H), 3.69 (s, 4H), 3.70-3.74 (m, 4H), 4.18-4.32 (m, 8H), 5.29-5.37 (m, 4H), 7.00-7.06 (m, 4H), 7.20-7.24 (m, 2H), 7.27-7.34 (m, 4H), 8.53-8.56 (m, 2H)

### [Example 20] Synthesis of Compound 20

### <Synthesis of Intermediate 25>

Using intermediate 6 (156 mg, 0.748 mmol) and 1-(3-aminoprop-1-yl)piperidine (48.4 mg, 0.340 mmol) and in the same manner as in Synthesis of intermediate 7 described in Example 2, 204 mg of intermediate 25 was synthesized.

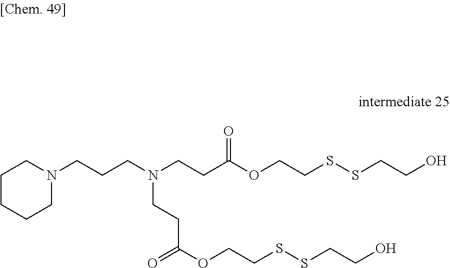 [SMILES: O=C(CCN(CCCN1CCCCC1)CCC(=O)OCCSSCCO)OCCSSCCO]

### <Synthesis of Compound 20>

Using 4-oleoyloxyphenylacetic acid (343 mg, 0.823 mmol) and intermediate 25 (204 mg, 0.365 mmol) and in the same manner as in Synthesis of compound 2 described in Example 2, 151 mg of compound 20 was synthesized.

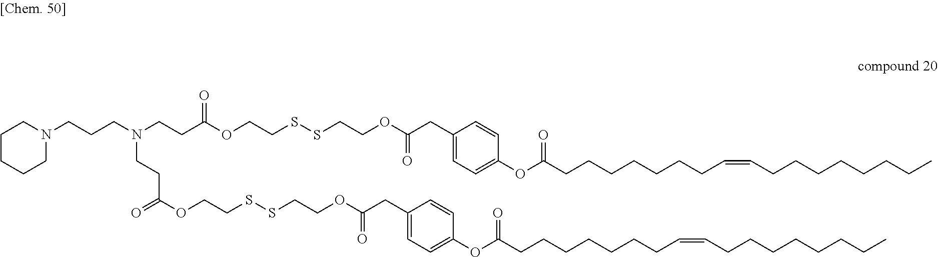 [SMILES: CCCCCCCC/C=C\CCCCCCCC(=O)Oc1ccc(CC(=O)OCCSSCCOC(=O)CCN(CCCN2CCCCC2)CCC(=O)OCCSSCCOC(=O)Cc2ccc(OC(=O)CCCCCCC/C=C\CCCCCCCC)cc2)cc1]

<¹H-NMR (600 MHz, DMSO-d₆) of Compound 20>

δ: 0.81-0.89 (m, 6H), 1.17-1.38 (m, 42H), 1.47-1.51 (m, 4H), 1.55-1.57 (m, 2H), 1.59-1.66 (m, 4H), 2.14-2.18 (m, 8H), 2.25-2.50 (m, 16H), 2.90-3.02 (m, 8H), 3.69 (s, 4H), 3.74-3.78 (m, 4H), 4.18-4.32 (m, 8H), 5.29-5.37 (m, 4H), 7.00-7.06 (m, 4H), 7.27-7.34 (m, 4H)

### [Example 21] Synthesis of Compound 21

### <Synthesis of Intermediate 26>

Using intermediate 6 (156 mg, 0.748 mmol) and 1-(2-aminoethyl)pyrrolidine (49.0 mg, 0.340 mmol) and in the same manner as in Synthesis of intermediate 7 described in Example 2, 205 mg of intermediate 26 was synthesized.

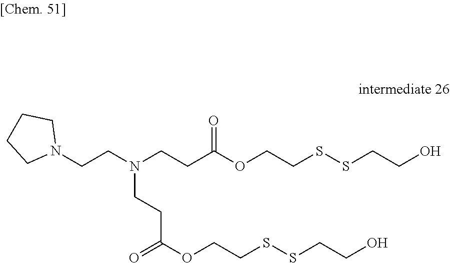 [SMILES: O=C(CCN(CCC(=O)OCCSSCCO)CCN1CCCC1)OCCSSCCO]

### <Synthesis of Compound 21>

Using 4-oleoyloxyphenylacetic acid (343 mg, 0.823 mmol) and intermediate 26 (205 mg, 0.386 mmol) and in the same manner as in Synthesis of compound 2 described in Example 2, 153 mg of compound 21 was synthesized.

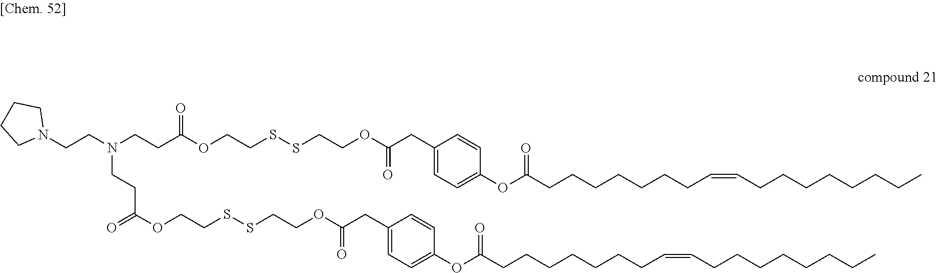 [SMILES: CCCCCCCC/C=C\CCCCCCCC(=O)Oc1ccc(CC(=O)OCCSSCCOC(=O)CCN(CCC(=O)OCCSSCCOC(=O)Cc2ccc(OC(=O)CCCCCCC/C=C\CCCCCCCC)cc2)CCN2CCCC2)cc1]

<¹H-NMR (600 MHz, DMSO-d₆) of Compound 21>

δ: 0.81-0.89 (m, 6H), 1.17-1.38 (m, 40H), 1.64-1.70 (m, 8H), 2.14-2.18 (m, 8H), 2.35-2.54 (m, 16H), 2.90-3.02 (m, 8H), 3.69 (s, 4H), 3.74-3.78 (m, 4H), 4.18-4.32 (m, 8H), 5.29-5.37 (m, 4H), 7.00-7.06 (m, 4H), 7.27-7.34 (m, 4H)

### [Example 22] Synthesis of Compound 22

Compound 22 was synthesized by the following synthesis route.

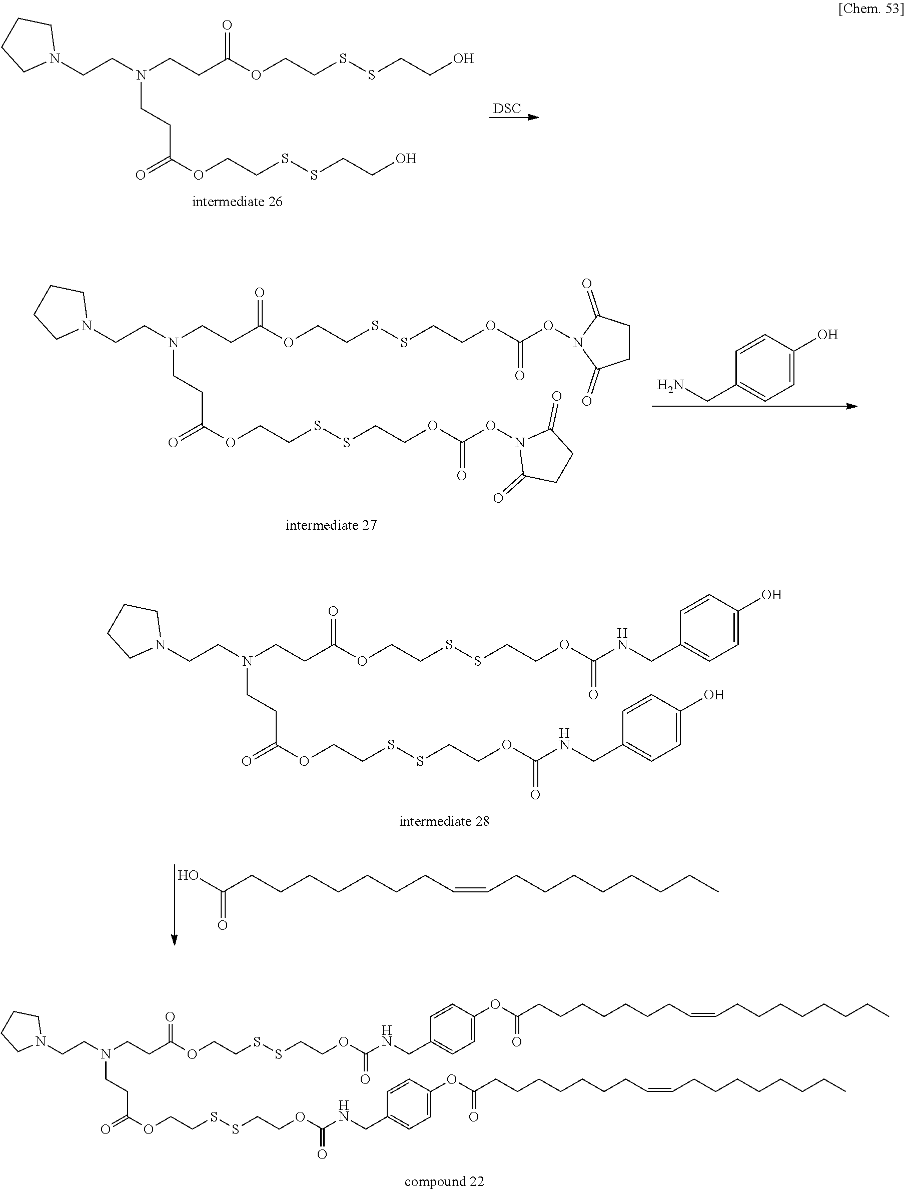

### <Synthesis of Intermediate 27>

Intermediate 26 (700 mg, 1.32 mnmol) was dissolved in dichloromethane (7.00 g), triethylamine (404 mg, 4.00 mnmol) and DSC (387 mg, 3.96 mmol) were added, and the mixture was reacted at room temperature for 1 hr. The solution after the reaction was filtered to give a filtrate containing intermediate 27.

### <Synthesis of Intermediate 28>

To the filtrate containing intermediate 27 were added 4-(aminomethyl)phenol (543 mg, 3.96 mmol) and triethylamine (404 mg, 4.00 mmol), and the mixture was reacted at room temperature for 4 hr. The solution after the reaction was washed with 5 wt % sodium dihydrogen phosphate aqueous solution and 20 wt % sodium chloride aqueous solution, and dehydrated with sodium sulfate. The filtrate after dehydration was concentrated using an evaporator. After concentration, silica gel column purification was performed using ethanol/chloroform to give 776 mg of intermediate 28.

### <Synthesis of Compound 22>

Using intermediate 28 (320 mg, 0.386 mmol) and oleic acid (240 mg, 0.849 mmol) and in the same manner as in Synthesis of 4-oleoyloxyphenylacetic acid described in Example 1, 338 mg of compound 22 was synthesized.

<¹H-NMR (600 MHz, DMSO-d₆) of Compound 22>

δ: 0.81-0.89 (m, 6H), 1.17-1.38 (m, 40H), 1.64-1.70 (m, 8H), 2.14-2.18 (m, 8H), 2.35-2.54 (m, 16H), 2.90-3.02 (m, 8H), 3.74-3.78 (m, 4H), 3.94-3.98 (m, 8H), 4.23 (s, 4H), 5.29-5.37 (m, 4H), 7.00-7.06 (m, 4H), 7.27-7.34 (m, 4H), 8.17-8.19 (m, 2H)

### [Example 23] Synthesis of Compound 23

Compound 23 was synthesized by the following synthesis route.

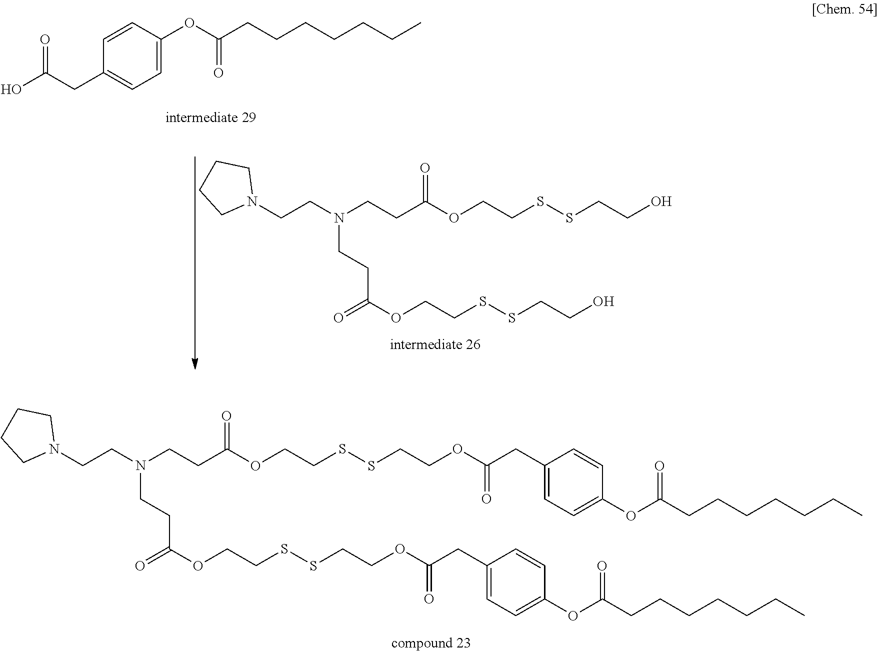 [SMILES: CCCCCCCC(=O)Oc1ccc(CC(=O)O)cc1 ; O=C(CCN(CCC(=O)OCCSSCCO)CCN1CCCC1)OCCSSCCO ; CCCCCCCC(=O)Oc1ccc(CC(=O)OCCSSCCOC(=O)CCN(CCC(=O)OCCSSCCOC(=O)Cc2ccc(OC(=O)CCCCCCC)cc2)CCN2CCCC2)cc1]

### <Synthesis of Intermediate 29>

Using n-octanoic anhydride (5.08 g, 18.8 mmol) and 4-hydroxyphenylacetic acid (2.60 g, 17.1 mmol) and in the same manner as in Synthesis of 4-oleyloxyphenylacetic acid described in Example 1, 3.26 g of intermediate 29 was synthesized.

### <Synthesis of Compound 23>

Using intermediate 29 (485 mg, 1.74 mol) and intermediate 26 (373 mg, 0.703 mmol) and in the same manner as in Synthesis of compound 2 described in Example 2, 316 mg of compound 23 was synthesized.

<¹H-NMR (600 MHz, DMSO-d₆) of Compound 23>

δ: 0.81-0.88 (t, 6H), 1.21-1.30 (m, 16H), 1.59-1.68 (m, 8H), 2.30-2.60 (m, 16H), 2.66-2.74 (m, 4H), 2.90-3.02 (m, 8H), 3.69 (s, 4H), 4.18-4.32 (m, 8H), 7.00-7.06 (m, 4H), 7.27-7.34 (m, 4H)

### [Example 24] Synthesis of Compound 24

Compound 24 was synthesized by the following synthesis route.

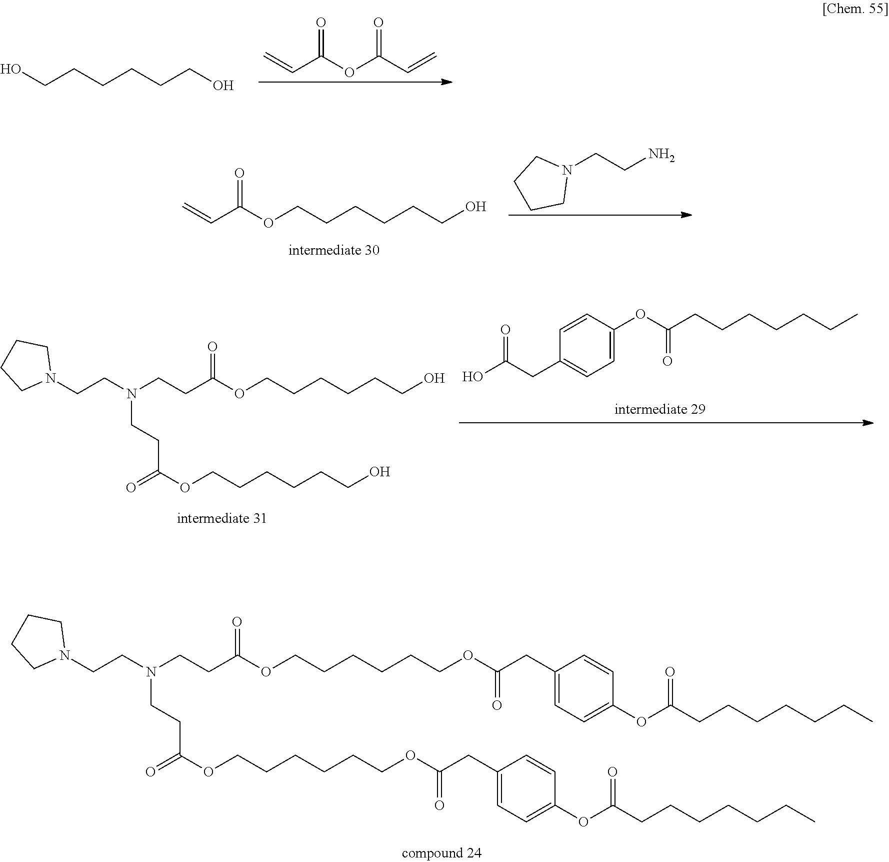

### <Synthesis of Intermediate 30>

Using 1,6-hexanediol (1.96 mg, 16.58 mmol) and acrylic anhydride (1.99 mg, 15.8 mmol) and in the same manner as in Synthesis of intermediate 6 described in Example 2, 913 mg of intermediate 30 was synthesized.

### <Synthesis of Intermediate 31>

Using 1-(2-aminoethyl)pyrrolidine (77.7 mg, 0.680 mmol) and intermediate 30 (258 mg, 1.50 mmol) and in the same manner as in Synthesis of intermediate 7 described in Example 2, 336 mg of intermediate 31 was synthesized.

### <Synthesis of Compound 24>

Using intermediate 31 (336 mg, 0.732 mmol) and intermediate 29 (521 mg, 1.87 mol) and in the same manner as in Synthesis of compound 2 described in Example 2, 314 mg of compound 24 was synthesized.

<¹H-NMR (400 MHz, DMSO-d₆) of Compound 24>

δ: 0.81-0.92 (t, 6H), 1.21-1.40 (m, 24H), 1.48-1.72 (m, 16H), 2.31-2.59 (m, 16H), 2.64-2.73 (m, 4H), 3.66 (s, 4H), 3.92-4.08 (m, 8H), 7.00-7.06 (m, 4H), 7.27-7.34 (m, 4H)

### [Example 25] Synthesis of Compound 25

Compound 25 was synthesized by the following synthesis route.

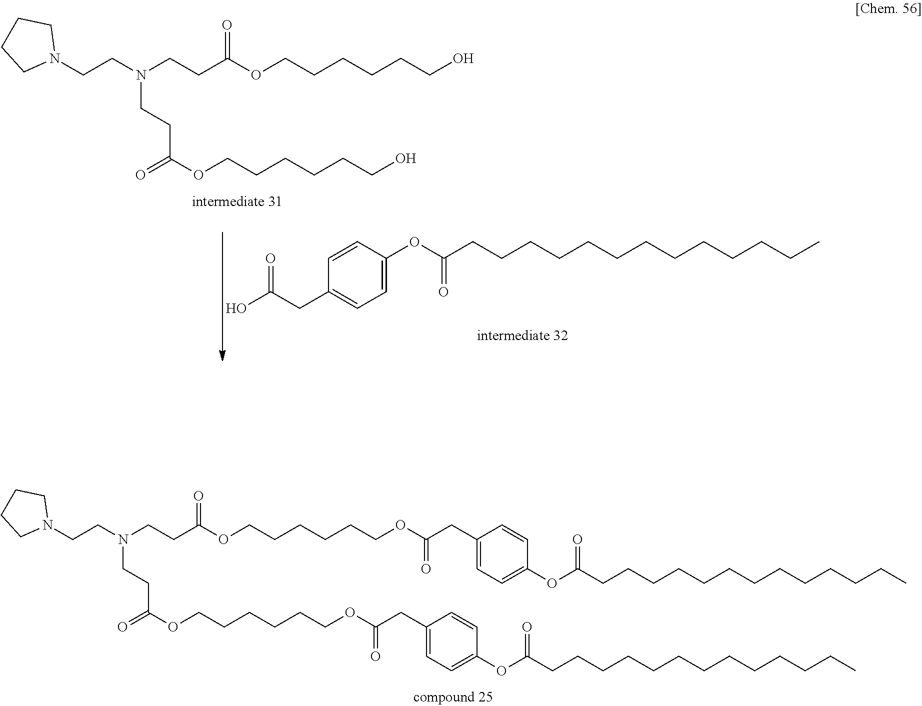 [SMILES: O=C(CCN(CCC(=O)OCCCCCCO)CCN1CCCC1)OCCCCCCO ; CCCCCCCCCCCCCC(=O)Oc1ccc(CC(=O)O)cc1 ; CCCCCCCCCCCCCC(=O)Oc1ccc(CC(=O)OCCCCCCOC(=O)CCN(CCC(=O)OCCCCCCOC(=O)Cc2ccc(OC(=O)CCCCCCCCCCCCC)cc2)CCN2CCCC2)cc1]

### <Synthesis of Intermediate 32>

Using myristic anhydride (2.19 g, 4.99 mnmol) and 4-hydroxyphenylacetic acid (1.73 g, 11.4 mmol) and in the same manner as in Synthesis of 4-oleyloxyphenylacetic acid described in Example 1, 2.03 g of intermediate 32 was synthesized.

### <Synthesis of Compound 25>

Using intermediate 31 (215 mg, 0.468 mmol) and intermediate 32 (417 mg, 1.15 mmol) and in the same manner as in Synthesis of compound 2 described in Example 2, 260 mg of compound 25 was synthesized.

<¹H-NMR (400 MHz, DMSO-d₆) of Compound 25>

δ: 0.81-0.92 (t, 6H), 1.21-1.44 (m, 52H), 1.50-1.71 (m, 16H), 1.79-1.93 (m, 4H), 2.41-2.51 (m, 4H), 2.55-2.77 (m, 8H), 3.71 (s, 4H), 3.98-4.11 (m, 8H), 7.06-7.11 (m, 4H), 7.27-7.34 (m, 4H)

### [Example 26] Synthesis of Compound 26

Compound 26 was synthesized by the following synthesis route.

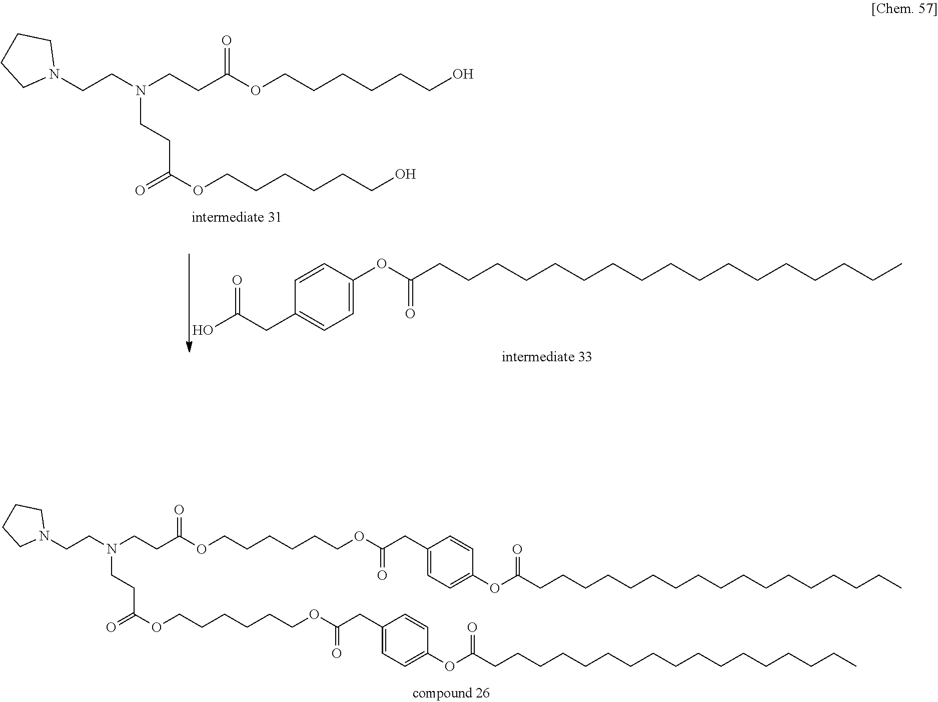 [SMILES: O=C(CCN(CCC(=O)OCCCCCCO)CCN1CCCC1)OCCCCCCO ; CCCCCCCCCCCCCCCCCC(=O)Oc1ccc(CC(=O)O)cc1 ; CCCCCCCCCCCCCCCCCC(=O)Oc1ccc(CC(=O)OCCCCCCOC(=O)CCN(CCC(=O)OCCCCCCOC(=O)Cc2ccc(OC(=O)CCCCCCCCCCCCCCCCC)cc2)CCN2CCCC2)cc1]

### <Synthesis of Intermediate 33>

Using stearic anhydride (2.07 g, 3.75 mmol) and 4-hydroxyphenylacetic acid (519 mg, 3.41 mmol) and in the same manner as in Synthesis of 4-oleyloxyphenylacetic acid described in Example 1, 1.50 g of intermediate 33 was synthesized.

### <Synthesis of Compound 26>

Using intermediate 31 (195 mg, 0.425 mmol) and intermediate 33 (402 mg, 0.96 mmol) and in the same manner as in Synthesis of compound 2 described in Example 2, 379 mg of compound 26 was synthesized.

### <¹H-NMR (400 MHz, Chloroform-d) of Compound 26>

δ: 0.83-0.91 (t, 6H), 1.21-1.44 (m, 64H), 1.50-1.68 (m, 20H), 1.69-1.80 (m, 4H), 2.41-2.58 (m, 8H), 2.71-2.83 (m, 4H), 3.60 (s, 4H), 3.98-4.11 (m, 8H), 7.00-7.04 (m, 4H), 7.21-7.32 (m, 4H)

### [Example 27] Synthesis of Compound 27

Using 4-oleoyloxyphenylacetic acid (401 mg, 0.963 mmol) and intermediate 31 (196 mg, 0.427 mmol) and in the same manner as in Synthesis of compound 2 described in Example 2, 399 mg of compound 27 was synthesized.

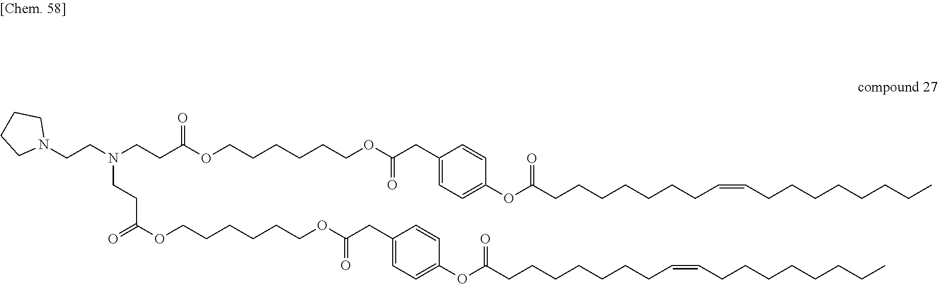 [SMILES: CCCCCCCC/C=C\CCCCCCCC(=O)Oc1ccc(CC(=O)OCCCCCCOC(=O)CCN(CCC(=O)OCCCCCCOC(=O)Cc2ccc(OC(=O)CCCCCCC/C=C\CCCCCCCC)cc2)CCN2CCCC2)cc1]

<¹H-NMR (600 MHz, DMSO-d₆) of Compound 27>

δ: 0.81-0.88 (t, 6H), 1.17-1.38 (m, 52H), 1.49-1.65 (m, 12H), 1.92-2.04 (m, 8H), 2.36-2.43 (m, 8H), 2.51-2.59 (m, 8H), 2.65-2.72 (m, 4H), 3.66 (s, 4H), 3.98-4.05 (m, 8H), 5.28-5.37 (m, 4H), 7.00-7.06 (m, 4H), 7.27-7.34 (m, 4H)

### [Example 28] Synthesis of Compound 28

Compound 28 was synthesized by the following synthesis route.

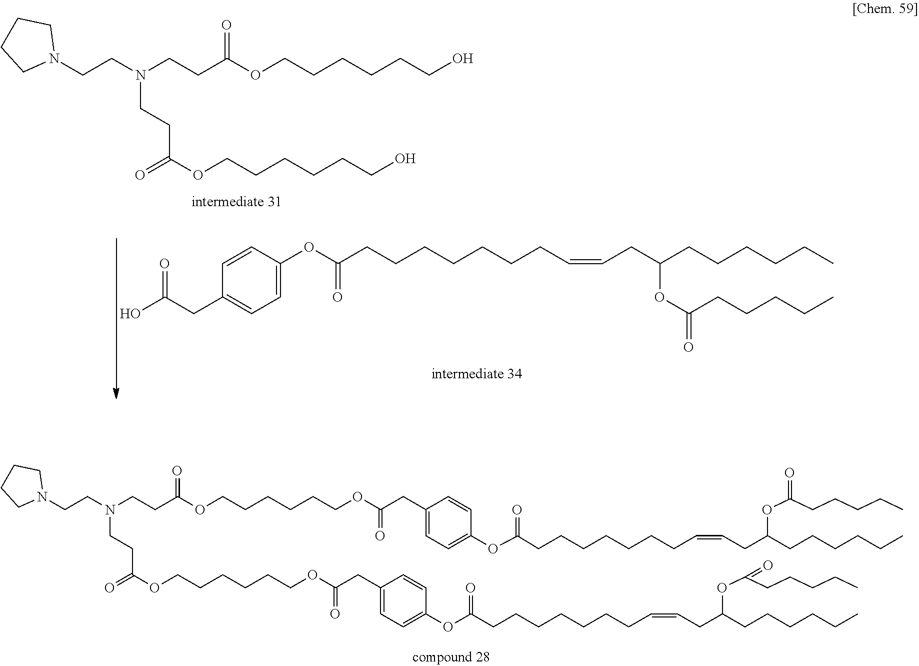 [SMILES: O=C(CCN(CCC(=O)OCCCCCCO)CCN1CCCC1)OCCCCCCO ; CCCCCCC(C/C=C\CCCCCCCC(=O)Oc1ccc(CC(=O)O)cc1)OC(=O)CCCCC ; CCCCCCC(C/C=C\CCCCCCCC(=O)Oc1ccc(CC(=O)OCCCCCCOC(=O)CCN(CCC(=O)OCCCCCCOC(=O)Cc2ccc(OC(=O)CCCCCCC/C=C\CC(CCCCCC)OC(=O)CCCCC)cc2)CCN2CCCC2)cc1)OC(=O)CCCCC]

### <Synthesis of Compound 28>

Using intermediate 31 (167 mg, 0.364 mmol) and intermediate 34 (433 mg, 0.816 mmol) synthesized according to the method described in WO 2024/203577 and in the same manner as in Synthesis of compound 2 described in Example 2, 343 mg of compound 28 was synthesized.

<¹H-NMR (600 MHz, DMSO-d₆) of Compound 28>

δ: 0.81-0.86 (t, 12H), 1.17-1.38 (m, 52H), 1.43-1.65 (m, 20H), 1.92-2.04 (m, 4H), 2.18-2.27 (m, 8H), 2.36-2.43 (m, 8H), 2.51-2.59 (m, 8H), 2.65-2.72 (m, 4H), 3.66 (s, 4H), 3.93-4.05 (m, 8H), 4.75-4.82 (m, 2H), 5.27-5.34 (m, 2H), 5.41-5.47 (m, 2H), 7.00-7.06 (m, 4H), 7.27-7.34 (m, 4H)

### [Example 29] Synthesis of Compound 29

Compound 29 was synthesized by the following synthesis

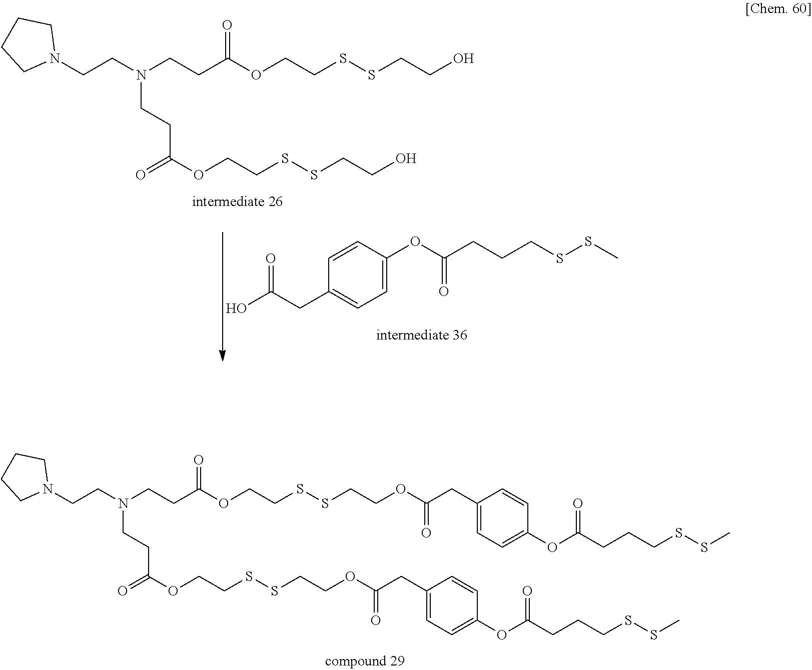 [SMILES: O=C(CCN(CCC(=O)OCCSSCCO)CCN1CCCC1)OCCSSCCO ; CSSCCCC(=O)Oc1ccc(CC(=O)O)cc1 ; CSSCCCC(=O)Oc1ccc(CC(=O)OCCSSCCOC(=O)CCN(CCC(=O)OCCSSCCOC(=O)Cc2ccc(OC(=O)CCCSSC)cc2)CCN2CCCC2)cc1]

### <Synthesis of Intermediate 35>

4-(Methyldisulfanyl)butanoic acid (3.80 g, 22.9 mmol) was dissolved in chloroform (38.0 g), DMAP (560 mg, 4.58 mmol) and EDC hydrochloride (6.58 g, 34.4 mmol) were added, and the mixture was reacted at room temperature for 2 hr. The solution after the reaction was concentrated using an evaporator. After concentration, silica gel column purification was performed using ethanol/chloroform to give 2.88 g of intermediate 35.

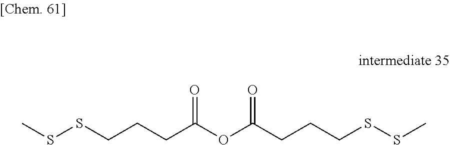

### <Synthesis of Intermediate 36>

Using intermediate 35 (2.88 g, 9.16 mmol) and 4-hydroxyphenylacetic acid (697 mg, 4.58 mmol) and in the same manner as in Synthesis of 4-oleyloxyphenylacetic acid described in Example 1, 1.10 g of intermediate 36 was synthesized.

### <Synthesis of Compound 29>

Using intermediate 26 (193 mg, 0.364 mmol) and intermediate 36 (241 mg, 0.801 mmol) and in the same manner as in Synthesis of compound 2 described in Example 2, 238 mg of compound 29 was synthesized.

<¹H-NMR (600 MHz, DMSO-d₆) of Compound 29>

δ: 1.64-1.70 (m, 4H), 1.96-2.00 (m, 4H), 2.30 (s, 6H), 2.35-2.39 (m, 4H), 2.47-2.54 (m, 16H), 2.90-3.02 (m, 8H), 3.69 (s, 4H), 3.74-3.78 (m, 4H), 4.18-4.32 (m, 8H), 7.00-7.06 (m, 4H), 7.27-7.34 (m, 4H)

### [Example 30] Synthesis of Compound 30

Using intermediate 31 (167 mg, 0.364 mmol) and intermediate 36 (241 mg, 0.801 mmol) and in the same manner as in Synthesis of compound 2 described in Example 2, 216 mg of compound 30 was synthesized.

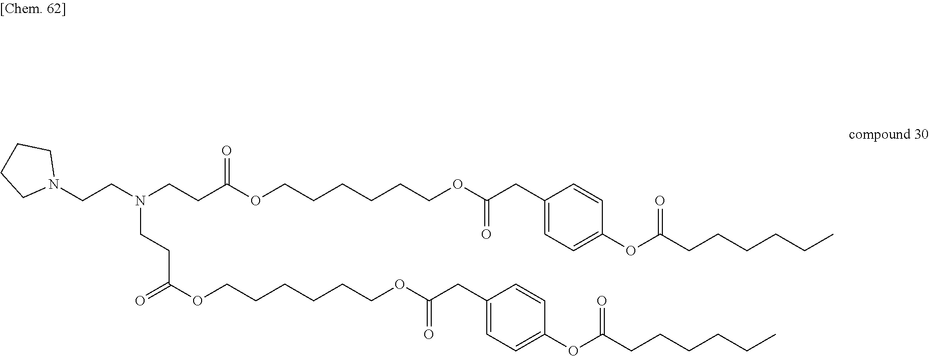 [SMILES: CCCCCCC(=O)Oc1ccc(CC(=O)OCCCCCCOC(=O)CCN(CCC(=O)OCCCCCCOC(=O)Cc2ccc(OC(=O)CCCCCC)cc2)CCN2CCCC2)cc1]

### <¹H-NMR (600 MHz, DMSO-d) of Compound 30>

δ: 1.41-1.45 (m, 8H), 1.58-1.62 (m, 8H), 1.64-1.70 (m, 4H), 1.96-2.00 (m, 4H), 2.30 (s, 6H), 2.35-2.39 (m, 4H), 2.47-2.54 (m, 16H), 3.69 (s, 4H), 3.74-3.78 (m, 4H), 4.18-4.32 (m, 8H), 7.00-7.06 (m, 4H), 7.27-7.34 (m, 4H)

### [Experimental Example 1] Measurement of Liposomal pKa

Lipid nano particles (LNP) without containing nucleic acid were used for the evaluation of Liposomal pKa.

### 1. Preparation of LNP by Micro Flow Path

### (1) Preparation of Ethanol Solution of Lipid

An ethanol solution of cationic lipid (5 mM), an ethanol solution of DOPC (3 mM), an ethanol solution of Chol (10 mM), and an ethanol solution of DMG-PEG2k (0.5 mM) were mixed at desired ratio (cationic lipid:DOPC:Chol:DMG-PEG2k=52.5:7.5:40:1.5 (molar ratio)) in an Eppendorf tube to achieve the total lipid amount of 800 nmol. Then, to the obtained mixture was added ethanol to prepare an ethanol solution (total amount: 1000 μL) of the lipid.

### (2) Preparation of LNP Using Micro Flow Path

An acidic malic acid buffer (20 mM, pH 5.0) (1080 μL) containing NaCl at a final concentration of 30 mM and an ethanol solution (360 μL) of lipid were each weighed in a syringe. Using an ultra high-speed nanomedicament producing apparatus NanoAssemblr (manufactured by Precision NanoSystems), LNP was prepared under the conditions of addition rate of acidic buffer solution: 3 mL/min, addition rate of ethanol solution of lipid: 1 mL/min, and syringe holder temperature: 25° C., and collected in a 15 mL tube. MES buffer (pH 6.5) (1000 μL) was added to the 15 mL tube, the obtained mixture was transferred to Amicon Ultra 4, and ultrafiltration was performed under centrifugation conditions (25° C., 1000 g, 6 min) to concentrate the mixture to about 100 μL. The obtained concentrate was diluted with PBS to 4 mL, and the mixture was concentrated again under centrifugation conditions (25° C., 1000 g, 6 min), and this operation was performed twice. The obtained concentrate was diluted with PBS to a lipid concentration of 0.5 mM to give a dispersion containing LNP.

### 2. Measurement of Liposomal pKa

20 mM Citrate buffer containing NaCl at a final concentration of 150 mM, sodium phosphate buffer, and tris HCl buffer, each adjusted to various pHs within the range of pH 3.0 to 10.0, were prepared. TNS (manufactured by Sigma) solution was diluted with ultrapure water to 0.6 mM. TNS solution (2 μL), dispersions (12 μL) containing LNP prepared in [Experimental Example 1], 1., and buffers adjusted to various pHs (186 μL) were added to a black 96 well plate. The plate was protected from light and shaken at 400 rpm for 10 min. The fluorescence intensity (excitation: 321 nm/emission: 447 nm) was measured using a plate reader (manufactured by TECAN). The relative fluorescence intensity was calculated as a percentage, with the maximum value of the fluorescence intensity in each LNP being 100% and the minimum value being 0%. Furthermore, the pH at which the relative fluorescence intensity was 50% was taken as Liposomal pKa. The Liposomal pKas of the cationic lipids and LNPs used are shown in Table 3.

TABLE 3

| cationic lipid used | Liposomal pKa |
|---|---|
| compound 1 | 5.28 |
| compound 3 | 7.82 |
| compound 6 | 4.86 |
| compound 7 | 5.66 |
| compound 27 | 7.65 |
| comparison compound | 6.44 |

### 3. Results

As shown in Table 3, LNPs containing compound 1, compound 3, compound 6, compound 7, compound 27, or comparison compound showed Liposomal pKa within the range (4.5 to 9.5) preferable for endosomal escape.

### [Experimental Example 2] Preparation of mRNA-Encapsulating Particles and Property Evaluation

### 1. Preparation of LNP by Micro Flow Path Method

### (1) Preparation of Ethanol Solution of Lipid

An ethanol solution (5 mM) of cationic lipid, an ethanol solution of DOPC (5 mM), an ethanol solution of Chol (10 mM), and an ethanol solution of DMG-PEG2k (0.5 mM) were mixed at desired ratio (cationic lipid:DOPC:Chol:DMG-PEG2k=50:10:40:1 (molar ratio)) in an Eppendorf tube to achieve the total lipid amount of 800 nmol. Then, to the obtained mixture was added ethanol to prepare an ethanol solution (total amount: 3067 μL) of the lipid.

### (2) Preparation of Acidic Buffer Solution of Nucleic Acid

An acidic buffer solution (total amount: 7480 μL) of nucleic acid was prepared by weighing 4.0 μg of mRNA solution (0.6 mg/mL) in a 5 mL tube and adding acidic malate buffer (20 mM, pH 3.0).

### (3) Preparation of LNP Using Micro Flow Path

An acidic buffer solution of nucleic acid and an ethanol solution of lipid were each weighed in a syringe. Using an ultra high-speed nanomedicament producing apparatus NanoAssemblr (manufactured by Precision NanoSystems), LNP was prepared under the conditions of addition rate of acidic buffer solution of nucleic acid: 3 mL/min, addition rate of ethanol solution of lipid: 1 mL/min, and syringe holder temperature: 25° C., and collected in a 15 mL tube. MES buffer (pH 6.5) (3000 μL) was added to the 15 mL tube, the obtained mixture was transferred to Amicon Ultra 4, and ultrafiltration was performed under centrifugation conditions (25° C., 1000 g, 6 min) to concentrate the mixture to about 500 μL. The obtained concentrate was diluted with PBS to 4 mL, and the mixture was concentrated again under centrifugation conditions (25° C., 1000 g, 6 min), and this operation was performed twice. The obtained concentrate was diluted with PBS to a lipid concentration of 2 mM to give a dispersion containing LNP.

2. Measurement of Particle Size, PdI and Zeta Potential of mRNA-Encapsulating LNPs

The particle size, PdI (Polydispersity Index), and zeta potential of the mRNA-encapsulating LNP prepared by the method of the above-mentioned 1 were measured by a dynamic light scattering method (Zetasizer Nano; Malvern). The cationic lipids used and the results are shown in Table 4.

TABLE 4

| cationic | particle | | zeta |
|---|---|---|---|
| lipid used | size (nm) | PdI | potential (mV) |
| compound 1 | 120 | 0.101 | −1.9 |
| compound 3 | 123 | 0.216 | 12.9 |
| compound 6 | 123 | 0.088 | −4.2 |
| compound 7 | 97 | 0.077 | −4.1 |
| comparison | 66 | 0.168 | −1.4 |
| compound | | | |

### 3. Results

As shown in Table 4, mRNA-encapsulating LNPs containing compound 1, compound 3, compound 6, compound 7, or comparison compound showed a more preferable particle size range of 30 to 200 nm, and the electric charge (zeta potential) thereof at physiological pH was within a preferable range (−15 mV to +15 mV).

### [Experimental Example 3] Preparation of mRNA-Encapsulating Particles and Property Evaluation

### 1. Preparation of LNP by Micro Flow Path Method

### (1) Preparation of Ethanol Solution of Lipid

A ethanol solution of cationic lipid (5 mM), an ethanol solution of DOPC (5 mM), an ethanol solution of Chol (10 mM), and an ethanol solution of DMG-PEG2k (0.5 mM) were mixed at desired ratio (cationic lipid:DOPC:Chol:DMG-PEG2k=40:10:48:2 (molar ratio)) in an Eppendorf tube to achieve the total lipid amount of 800 nmol. Then, to the obtained mixture was added ethanol to prepare an ethanol solution (total amount: 3200 μL) of the lipid.

### (2) Preparation of Acidic Buffer Solution of Nucleic Acid

An acidic buffer solution (total amount: 15.6 mL) of nucleic acid was prepared by weighing 4.0 μg of mRNA solution (0.6 mg/mL) in a 5 mL tube and adding acidic citrate buffer (20 mM, pH 5.0).

### (3) Preparation of LNP Using Micro Flow Path

An acidic buffer solution of nucleic acid and an ethanol solution of lipid were each weighed in a syringe. Using an ultra high-speed nanomedicament producing apparatus NanoAssemblr (manufactured by Precision NanoSystems), LNP was prepared under the conditions of addition rate of acidic buffer solution of nucleic acid: 3 mL/min, addition rate of ethanol solution of lipid: 1 mL/min, and syringe holder temperature: 25° C., and collected in a 15 mL tube. Tris-buffered saline (pH 7.6) (3000 μL) was added to the 15 mL tube, the obtained mixture was transferred to Amicon Ultra 4, and ultrafiltration was performed under centrifugation conditions (25° C., 1000 g, 6 min) to concentrate the mixture to about 500 μL. The obtained concentrate was diluted with Tris-buffered saline to 4 mL, and the mixture was concentrated again under centrifugation conditions (25° C., 1000 g, 6 min), and this operation was performed twice. The obtained concentrate was diluted with Tris-buffered saline to a lipid concentration of 2 mM to give a dispersion containing LNP.

2. Measurement of Particle Size, PdI and Zeta Potential of mRNA-Encapsulating LNP

The particle size, PdI (Polydispersity Index), and zeta potential of the mRNA-encapsulating LNP prepared by the method of the above-mentioned 1 were measured by a dynamic light scattering method (Zetasizer Nano; Malvern). The cationic lipid used and the results are shown in Table 5.

TABLE 5

| cationic | particle | | zeta |
|---|---|---|---|
| lipid used | size (nm) | PdI | potential (mV) |
| compound 27 | 115 | 0.152 | 5.22 |

### 3. Results

As shown in Table 5, mRNA-encapsulating LNP produced using compound 27 as a cationic lipid showed a preferable particle size range of 30 to 300 nm, and the electric charge (zeta potential) thereof at physiological pH was within a preferable range (−15 mV to +15 mV).

### [Experimental Example 4] Evaluation of In Vitro Gene Expression in HeLa Cells

1. Preparation of mRNA-Encapsulating LNP

Using compound 3, compound 7, or comparison compound, LNP encapsulating mRNA that expresses luciferase was prepared by the method described in [Experimental Example 2], 1. In addition, using compound 27, LNP encapsulating mRNA that expresses luciferase was prepared by the method described in [Experimental Example 3], 1.

### 2. Time-Course Evaluation of Gene Expression in HeLa Cells

HeLa cells, which are human cervical cancer cells, were seeded in a 3.5 cm dish at 5.0×10⁴ cells/2 mL/Dish 24 hr before transfection. After 24 hr, the medium was changed to a culture medium (D-MEM) containing 0.1 mM D-luciferin. The prepared mRNA-encapsulating LNP was diluted with PBS or Tris-buffered saline to an mRNA concentration of about 1 μg/mL. The diluted mRNA-encapsulating LNP solution (about 200 μL, mRNA: 0.2 μg) was added to a 3.5 cm dish, and set in an incubator luminometer KronosDio. The luminescence intensity of luciferase was measured for 2 min every one hour. The cumulative luciferase luminescence intensity in 24 hr was calculated from the obtained time change of expression. Cationic lipids used, cumulative luciferase luminescence intensity in 24 hr, and relative luminescence intensity (=cumulative luciferase luminescence intensity in 24 hr when cationic lipid of Example is used/cumulative luciferase luminescence intensity in 24 hr when comparison compound is used) are shown in Table 6. “E+0a” (a: integer) indicated in Table 6 means “10^{a}”. For example, “5.19E+08” means “5.19×10⁸”.

TABLE 6

| | cumulative luciferase | relative |
|---|---|---|
| cationic | luminescence intensity | luminescence |
| lipid used | (RLU) in 24 hr | intensity |
| compound 3 | 5.19E+08 | 58.6 |
| compound 7 | 3.46E+07 | 3.91 |
| compound 27 | 1.47E+08 | 16.6 |
| comparison | 8.85E+06 | 1.000 |
| compound | | |

### 3. Results

The higher the cumulative luciferase luminescence intensity in 24 hr, i.e., the higher the total luciferase activity is, the higher the gene expression is. As shown in Table 6, LNPs containing compound 3, compound 7, or compound 27 exhibited superior gene expression activity compared to the LNP containing the comparison compound. Therefore, it was clarified that the LNP containing the cationic lipid of the present invention is beneficial as an LNP with good nucleic acid delivery efficiency.

### INDUSTRIAL APPLICABILITY

The cationic lipid of the present invention is useful for nucleic acid medicaments, gene therapy, biochemical experiments, and the like.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A cationic lipid represented by the formula (1): 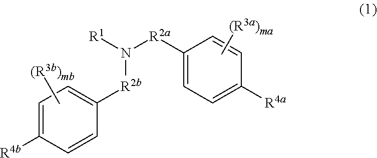
wherein
R¹ is an alkyl group having 1 to 4 carbon atoms and a nitrogen-containing heterocyclic group, a tertiary amino group, or a hydroxy group as a substituent,
R^{2a} and R^{2b} are each independently an alkylene group having 1 to 16 carbon atoms and containing at least one degradable bond,
R^{3a} and R^{3b} are each independently an alkoxy group having 1 to 4 carbon atoms or a halogen atom,
ma and mb are each independently an integer of 0 to 4,
R^{4a} and R^{4b} are each independently R^{S}—CO—O—* (wherein * is a bonding position), and
R⁵ is a monovalent aliphatic hydrocarbon group having 3 to carbon atoms and optionally containing at least one degradable bond, or R⁶—CO— (CH₂)ₚ—* (wherein * is a bonding position, R⁶ is a residue of a liposoluble vitamin having a hydroxyl group or a residue of a sterol derivative having a hydroxyl group, and p is an integer of 1 to 8).

2. The cationic lipid according to claim 1, wherein the nitrogen-containing heterocyclic group is a pyrrolidinyl group, a morpholinyl group, a piperazinyl group, a piperidyl group, an azepanyl group, a pyrrolyl group, a pyrazolyl group, a pyridyl group, or an indolyl group,
the tertiary amino group is a di(alkyl)amino group, and
the carbon numbers of the two alkyl groups in the di(alkyl)amino group are each independently 1 to 4.

3. The cationic lipid according to claim 1, wherein the R^{2a} and R^{2b} are each independently a divalent group represented by the formula (2): 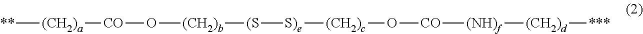
wherein
** is a bonding position to the nitrogen atom in the formula (1),
*** is a bonding position to the carbon atom of the benzene ring in the formula (1),
a to d are each independently an integer of 1 to 4, and
e and f are each independently 0 or 1.

4. The cationic lipid according to claim 1, wherein ma and mb are each 0.

5. The cationic lipid according to claim 1, wherein R⁵ is a monovalent aliphatic hydrocarbon group having 3 to 30 carbon atoms and optionally containing at least one degradable bond.

6. A lipid membrane structure comprising the cationic lipid according to claim 1 as a constituent lipid of the membrane.

7. The lipid membrane structure according to claim 6, further comprising a nucleic acid.

8. A nucleic acid-introducing agent comprising the cationic lipid according to claim 1.

9. The nucleic acid-introducing agent according to claim 8, further comprising a nucleic acid.

10. A pharmaceutical composition comprising the cationic lipid according to claim 1.

11. The pharmaceutical composition according to claim 10, further comprising a nucleic acid.

12. A method for introducing a nucleic acid in a nucleic acid-introducing agent into a cell in vitro, comprising bringing the nucleic acid-introducing agent according to claim 9 into contact with the cell.

13. A method for introducing a nucleic acid in a nucleic acid-introducing agent into a target cell in a living organism, comprising administering the nucleic acid-introducing agent according to claim 9 to the living organism.

14. A method for producing a cellular medicine comprising a cell expressing a gene in a nucleic acid, comprising introducing the nucleic acid in a nucleic acid-introducing agent into a cell by bringing the nucleic acid-introducing agent according to claim 9 into contact with the cell.
